(19) United States
(12) Patent Application Publication
(10) Pub. No.: US 2007/0219213 A1
(43) Pub. Date: Sep. 20, 2007

(54) Use of Adrenergic N-Phenylpiperazine Antagonists, Pahrmaceutical Compositions Containning Them, and Methods of Preparing Them

(76) Inventors: Luiz Antonio Soares Romeiro, Basil (BR); Carlos Alberto Manssour Fraga, Rio de Janeiro (BR); Eliezer de Jesus De Lacerda Barreiro, Rio de Janeiro (BR); Clardia Lucia Martins da Silva, Rio de Janeiro (BR); Ana Luisa Palhares Miranda, Rio de Janeiro (BR); Eduardo Vera Tibirica, Rio de Janeiro (BR); Fernanda Carla Ferreira de Brito, Rio de Janeiro (BR); Helena Carla Castro Cardoso de Almeida, Rio de Janeiro (BR); Francois Germain Noel, Rio de Janeiro (BR)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 11/569,025

(22) PCT Filed: May 20, 2005
(86) PCT No.: PCT/BR05/00090

§ 371 Date: Nov. 13, 2006

(30) Foreign Application Priority Data
May 20, 2004 (BR) PI 040.1797-8

(51) Int. Cl. A61K 31/495; C07D 241/04

(52) U.S. Cl. 514/252.12; 544/358

(57) ABSTRACT

The present invention describes phenylpiperazinyl alpha adrenergic antagonists that corresponds to the formula (I) which selectively act on the alpha 1A/alpha 1D subtypes, where its selectivity index in comparison to alpha 1B subtype is, at minimum, 1700 for the alpha 1A subtype and 10000 for the alpha 1 D subtype, being therefore useful for the treatment of the lower urinary tract symptoms, including the benign prostatic hyperplasia treatment in mammals, preferentially humans. It is also described pharmaceutical compositions containing said compounds and methods for its preparation.

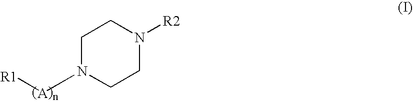

### FIELD OF THE INVENTION

The present invention is related to N-phenylpiperazine derivatives. Particularly, the present invention relates to (3,4-methylenedioxyphenyl) alkyl-N-phenylpiperazine and phenylalkyl-N-phenylpiperazine derivatives, substituted or not, and their isosteres, a method of preparing them, pharmaceutical compositions containing them and their use as (alpha)1-adrenoceptor antagonist therapeutic agents, particularly in the treatment for benign prostatic hyperplasia.

### BACKGROUND OF THE INVENTION

The human adrenergic receptors are members of the G protein-coupled transmembrane receptor superfamily and were classified in two groups: alpha (alpha-AR) and beta (beta-AR) adrenoceptors. Both types of receptors have norepinephrine and epinephrine biogenic amines as endogenous agonists, and modulate the actions of these catecholamines in the peripheral sympathetic nervous system.

Norepinephrine is produced by adrenergic nerve terminations, while epinephrine is produced by adrenal marrow. The adrenergic receptors' affinity for these compounds is the basis of their classification: the alpha receptors have high affinity for norepinephrine in relation to epinephrine, and considerably higher affinity if compared to the synthetic compound isoproterenol.

Subsequently, the functional distinction between alpha and beta adrenoceptors was elucidated and refined, based on the characterization of these receptors from several animals and tissue sources. As result of these investigations, the alpha and beta adrenoceptors were afterwards subdivided into alpha 1, alpha 2, beta 1 and beta 2. The functional differences between subtypes alpha 1 and alpha 2 have been evidenced by the development of selective ligands for these receptors.

More about the alpha 1 adrenergic receptors (alpha 1-AR) can be found in Robert R. Ruffolo: alpha-adrenoceptor: Molecular Biology, Biochemistry and Pharmacology (Progress in Basic and Clinical Pharmacology series, Karger, 1991), which explores topics related to the foundations of the alpha 1/alpha 2 sub-classification, molecular biology, signal transduction, relationships between chemical structure and biological activity for agonists, receptor function, and therapeutic applications for compounds that present affinity for the adrenergic receptors.

The cloning, sequencing and expression of the alpha 1-AR subtypes from animal tissues (Bruno et al., Biochem. Biophys. Res. Commun. 179:1485-1490, 1991; Forray et al., Mol. Pharmacol. 45: 703-708, 1994; Hirasawa et al., Biochem. Biophys. Res. Commun. 195: 902-909, 1993; Ramarao et al., J. Biol. Chem. 267: 21936-21945, 1992; Schwinn et al., J. Pharmacol. Exp. Ther. 272: 134-142, 1995; Weinberg et al., Biochem. Biophys. Res. Commun. 201: 1296-1304, 1994) resulted in sub-classification of this receptor family to alpha 1 D (formerly named alpha 1A or alpha 1A/1C), alpha 1B and alpha 1A (formerly named alpha 1C). Recently, a fourth subtype of this receptor family, named alpha 1L (due to the comparatively low affinity in comparison to the antagonist prazosin, selective for the alpha 1 adrenoceptors sub-family was described by Muramatsu (Muramatsu et al., Br. J. Urol. 74: 572-578, 1994). However, this new subtype, which would be involved in the prostatic smooth muscle contraction in humans, was not cloned yet, because the absence of mRNA that codes it, suggesting evidence that this represents a low affinity state of the (alpha)1-adrenoceptor, and not a distinct receptor (Ford et al., Mol. Pharmacol. 49: 209-215, 1997). Each one of the receptor subtypes of the alpha 1 subfamily presents specificity regarding the pharmacology and tissue expression. The name alpha 1A is a recommendation recently approved by IUPHAR Nomenclature Committee for the cloned subtype previously designated alpha 1C published in the Receptor and Ion Channel Nomenclature Supplement (Watson & Girdlestone, 1995). The name alpha 1A, heretofore used in this invention, refers to this adrenoceptor subtype. At the same time, the nomenclature referred previously to the alpha 1A subtype was renamed to alpha 1D and heretofore will be used in this invention. A lineage of stable cells was deposited into the American Type Culture Collection (ATCC) under the old nomenclature. For review on the alpha 1 adrenoceptors subfamliy classification, see Martin et al. Naunyn-Schmiederesberg's Arch. Pharmacol, 352:1-10, 1995; Zhong & Minneman Eur. J. Pharmacol, 375:261-276, 1999; Michelotti et al. Pharmacol. Ther. 88: 281-309, 2000.

The differences among alpha 1-AR receptor subtypes have been important in the clinical treatment of several physiopathologies in which the hypertension and the obstructive symptoms of the lower urinary tract flow are evident. This has been compelling the search for the identification and characterization of new receptors, as well as putative therapeutically useful molecules.

Particularly for the genitourinary system, several studies have characterized the presence of these receptors in the lower urinary tract, particularly in human prostatic tissue and in detrusor of the bladder (Forray et al, Mol. Pharmacol. 45: 703-708, 1994, Hatano et al., Br. J. Pharmacol. 113: 723-728, 1994; Marshall et al., Br. J. Pharmacol. 107 (proc. Suppl. Dec.): 327P, 1992, Marshall et al., Br. J. Pharmacol. 115: 243-247, 1995; Yamada et al., Life Sci. 54: 1845-1854, 1994, Muramatsu et al., Br. J. Urol. 74: 572-578, 1994; Hieble et al., Pharmacol. Rev. 47: 267-270, 1995; Michelotti et al., Pharmacol. Ther. 88: 281-309, 2000).

The prostate is the gland responsible for the production of the spermatic liquid, secretion that, together with products from the seminal vesycles and periurethral glands, constitute sperm, liquid expelled during the ejaculation. The prostatic liquid participates in the nutrition and preservation of the spermatozoids produced in the testicles, in addition to containing espermina, substance that participates in the sperm liquefaction.

The micturition results from the detrusor muscle contraction, that consists of a tangle of smooth muscle fiber under autonomous sympathetic control from the sacral-spinal cord. A stimulus reflex is formed by sensory nerves for pain, temperature and distention, which leave the bladder to the sacral cord. However, sensory regions also reach the micturition center (PMC), resulting in generation of nerve impulses that usually suppress the sacral-spinal reflex arc, controlling the bladder emptying. In this meaning, the normal micturition is initiated by a voluntary suppression of the arc reflex cortical inhibition and by muscle relaxation of the pelvic colon and outer sphincter. Finally, the detrusor muscle contracts, and bladder emptying occurs.

Anomalies in the lower urinary tract e.g. dysuria, incontinence and enuresis, are common for the population in general. The dysuria includes urinary frequency, nocturia and urgency, and can be caused by cystitises, prostitis or benign prostatic hyperplasia, or neural disorders. The incontinence syndromes include stress, urgency and excessive flow incontinence. But the enuresis is related to involuntary passage of the urine at night or during sleep.

The benign prostatic hyperplasia, also known as benign prostatic hypertrophy or BPH, is the anatomopathologic term for the prostatic alterations represented by clinical, morphologic and functional changes in the urinary tract. The benign prostatic hyperplasia consists of nonmalignant enlargement of the prostate, being the benign tumor more commonly found in men.

It is estimated that 50% of the men with age greater than 50 years will show BPH related symptoms; from these, 20% to 30% will present obstruction of the urinary flow created by the prostatic growth (Hieble & Caine, Fed. Proc. 45: 2601-2603, 1986) and will need surgical treatment. BPH prevalence increases with aging, and jumps to approximately 50% in men of around 60 years, reaching 80% of the masculine population at the age of 80 (Cockett et al., The Second International Consultation on Benign Prostatic Hyperplasia. Channel Island, Scientific Communication International Ltd, p 131, 278, 281, 284, 554-559, 1994).

In general, the secondary obstruction to BPH occurs dependent on two factors: a static component, represented by the hyperplasic glandular tissue, related to the gland prostatic enlargement, which can result in urethra compression and obstruction of the urinary flow from the bladder; and a dynamic component resultant from the prostatic stroma smooth musculature contraction dependent on predominantly adrenergic enervation and regulated by alpha 1 adrenoceptors.

Regarding the diagnosis, they are countless studies concerning mictiritional symptoms and the therapeutic options in BPH. Many aspects are important for the determination of the real BPH influence on patient's symptomatology and the choice of method of adequate treatment, once the symptoms of the urinary tract are not specific and, perhaps, the fundamental point in the diagnosis is to relate these symptoms to the presence of infravesical obstruction created by BPH, considering that all the methods of treatment, clinical or surgical, aim to decrease this condition.

The most serious problem in BPH can result in the need for surgical interventions. In these cases, the exeresis of prostatic hyperplastic tissue can be performed through several techniques, with transurethral, suprapubic, perineal approaches. Among the options addressed for the surgical treatment of the BPH static component, is the transurethral resection of the prostate (TURP), open prostatectomy, suprapubic prostatectomy, minimally invasive treatments, laser transurethral prostatectomy, hyperthermia and microwave thermotherapy, high intensity ultra-sound and transurethral ablation of the prostate with needle or laser.

The transurethral resection of the prostate (TURP), corresponds to the most popular surgical technique, with about 350 thousand procedures performed annually in the United States of America (Holtgrewe et al. J Urol 141: 248-252, 1989.), representing the second most performed surgical procedure by public health services, being overtaken only by cataract surgery, with an approximate cost greater than two billion dollars per year (Weis et al., Prostate 22: 325-334, 1993). In general, it is based on the hyperplasic tissue resection, using a resectoscope connected to an electrocautery, having for reference points the vesical colon and verumontanum, the later of importance in the lesion prevention of the outer urinary sphincter. The TURP morbidity is approximately 18% and the mortality varies around 0.2% (Mebust et al. J Urol 141: 243-246, 1989), both increasing with age, particularly above 80 years. The most important intraoperative complication corresponds to the syndrome of water intoxication, that results from exceeded absorption by the crude prostatic bed, of the nonionic and hypotonic solution used for vesical irrigation during the procedure. In this eventuality, that occurs in about 2% of the procedures, the decrease of the plasmatic osmolarity determines hemolysis occurrence, hyponatremia and arterial hypertension, that can result in acute renal insufficiency and convulsions (Mebust W. et al. J Urol 141: 243-246, 1989).

The late complications include incontinence, that occurs in up to 2-4% of the cases, being secondary to the outer urinary sphincter lesion or to the non diagnosed detrusor hyperactivity (Khan et al. Urology 38: 483-487, 1991), and the contracture of vesical colon secondary to postoperative fibrosis, that can occur in up to 1.7%. Erectile dysfunction can occur in up to 13.6% of the cases (McConnell J. et al., Benign prostatic hyperplasia: Diagnosis and treatment. Clinical Practice Guidelines, no. 8. AHCPR Publication no. 94-0582. Rockville, Md., Agency for Health Care Policy and Research, Public Health Service, US Department of Health and Human Services, 1994), secondary thermal lesion of the cavernous nerves that run sidelong to the prostatic capsule, or of the blood supply of the cavernous body, provoked by the electrocautery. Other possible complications include aspermia and retrograde ejaculation (70-75%).

Prolonged studies describe resolution of symptoms in up to 80% to 90% of the cases after one year from the resection, with decrease to 60% to 75% of patients after five years (Roos et al. N Engl J Med 320: 1120, 1986). In adequately selected patients, TURP nowadays corresponds to the procedure with larger probability for the resolution of the prostatic symptoms, corresponding to the standard treatment against which all emerging options of treatment have been compared.

As an alternative to surgical procedures, some pharmacological treatments are available, being, usually, geared to the dynamic component of this physiopathology. However, besides the availability of some molecules with therapeutic potential, the search for new molecules continues, notably in the problems presented by currently available molecules, reported as follows.

Selective antiandrogens are represented mostly by finasteride (Proscar® RTM, Merck). The use of antiandrogens in the treatment of BPH was proposed from old evidence on the improvement of the symptoms after orchiectomy. In the prostate, usually occurs the conversion of testosterone into dihydroxytestosterone, that corresponds to the metabolically active fraction in the prostatic tissue, mediated by the enzyme 5 alpha-reductase. The finasteride acts as a competitive antagonist of this enzyme. Controlled clinical studies demonstrated the reduction of the prostatic volume of about 20% to 30% and improvement of the urinary flow, being results significantly superior to placebo (McConnell et al., Hormonal treatment of benign prostatic hyperplasia. In Cockett, Khoury S, Aso Y et al. (Eds): Proceedings of the 2nd International Consultation on Benign Prostatic Hyperplasia. Paris, Scientific Communication International Ltd. P. 418, 1993.). However, in open population studies, the benefit magnitude is modest, and approximately one third of patients referred to some degree of symptomatology improvement (Stoner And:, Arch. Intern. Med. 154: 83-87, 1994). Until now, the capacity of finasteride to prevent the long term increase of prostate volume was not determined. Their side effects include erectile dysfunction and libido decrease in up to 5% of the patients (Stoner And:, Urology 43: 284-289, 1994). The impact of the decrease of the prostate-specific antigen (PSA) by this agent, on the early diagnosis for prostate cancer, had not been determined yet, and patients should be informed to this respect before the beginning of the treatment (Gormley et al., N Engl J Med 327: 1185, 1992).

Other therapeutic goals associated with the relief of obstructive symptoms characteristic of BPH have been the use of antagonist agents of adrenergic receptors (alpha 1-AR blockers), that act in the decrease of prostatic muscular tonus, due to the elevated concentration of these receptors in this tissue, particularly the subtypes alpha 1A and alpha 1B, being the first the predominant subtype (Lepor et al., Prostate 22: 301-311, 1993).

A variety of alpha 1-adrenergic antagonists have been investigated for the treatment of BPH. Among agents of current therapeutic use, there are the quinazoline derivatives as prazosin (Minipress®, Pfizer), terazosin (Hytrin®, Abbott), doxazosin mesilate (Cardura®, Pfizer) and alfuzosin (Xatral®, Sanofi, described in the patent EP 0204597, incorporated here as reference), which are nonselective alpha 1 antagonists. In view of non selectivity in comparison to the alpha 1 receptor subtypes, these agents present side effects on the peripheric vascularization e.g. syncope, asthenia, vertigo; and hypotension—the more commonly evidenced adverse effect (Lepor et al. J. Urol. 148: 1467-1474, 1992).

These adverse effects, caused by the nonselective antagonism on both prostatic and vascular tissues, have been a challenge in the planning of new selective antagonists for the treatment of BPH (uroselectivity). Among the alpha 1-AR antagonists approved for clinical utilization, besides quinazoline derivatives, tamsulosin has presented a better uroselectivity profile, with reduced pressure effects. This substance, known also by Flomax® (Boehringer Ingelheim) is described in patents U.S. Pat. No. 4,703,063; U.S. Pat. No. 4,731,478 U.S. Pat. No. 4,772,475 and U.S. Pat. No. 4,868,216, incorporated herein as reference. However, since its modest selectivity regarding alpha 1 adrenoceptor subtypes, its uroselectivity seems to be limited to the therapeutic dosage (0.4 mg/day).

The alpha 1-AR blocking agents present faster therapeutic effects In comparison to the 5 alpha-reductase enzyme inhibitors. However this effect, relative to the improvement of the obstructive symptoms and to the speed of the urinary flow, is considered moderate (Oestherling, N Engl J Med 332: 99-109, 1995).

Additionally, combinations of 5 alpha-reductase enzyme inhibitors with alpha 1 adrenoreceptor antagonists (prazosin, terazosin, doxazosin, alfuzosin, bunazosin, indoramine) have been described (WO 92/161213), in the attempt to act synergistically in the relief of the obstructive symptoms (alpha 1-AR) and prostate size reduction (5 alpha-reductase inhibitors).

Most alpha 1-AR antagonists presents a general model of structural attributes, necessary for the alpha 1 receptor recognition, which considers, at least, the presence of an atom of basic nitrogen to a distance of 5.0-7.5 Å from the aromatic ring centroid (A). This nitrogen atom, in the ion ammonium form, is responsible for the primary recognition by the receptor, considered as the main pharmacophoric group, that ionically interacts with the carboxylate group of Asp113 residue in the third transmembrane helix (TM3).

The aromatic ring has an important structural attribute, through the electronic contributions of their pi electrons and its hydrophobicity in pi-stacking interactions with hydrophobic amino acid residues (TM2, TM4, TM6, TM7). Additionally, the presence of a second aromatic ring (B) or heterocycle, e.g. amides, imides, should contain the presence of hydrogen-bonding acceptor groups (HBA) to a distance of 4.0-7.7 Å, from the ammoniacal nitrogen. Specifically for the aromatic ring, these HBA groups, preferentially occupy the positions meta and para, in relation to the spacer.

Another characteristic of great relevance for the affinity modulation of these antidrenergic compounds is the substitution pattern of the aromatic ring (A), including a) substitutions in the position ortho by groups that present negative electrostatic potential that are favorable for the affinity; b) the position meta of the same subunit seems to be involved in the selectivity, once the steric requirements for the alpha 1 receptor are more restricted, exhibiting an optimized Van der Waals value between 11-25 Å³; c) the position para represents a region where the ligand volume is limited, allowing the access and accommodation of small substitutes, like fluorine, in the bioreceptor cavity.

Finally, the size, nature and the conformation flexibility of the atom chain that forms the spacer, among primary (ammoniacal nitrogen) and secondary (substituted aromatic rings) pharmacophoric units, constitute other important structural characteristic, having influence not only in the affinity modulation, but also on the selectivity against other metabothropic protein G-coupled receptors e.g. 5-HT1A, D2.

Recent studies demonstrated that only the alpha 1D and alpha 1A (alpha 1D>alpha 1A) subtypes are expressed in human detrusor (Malloy et al., J. Urol. 160: 937-943, 1998) and suggest that the antagonism of alpha 1D subtype would be related to the relief of the irritating symptoms of bladder flow obstruction e.g. urgency, nocturia, and bladder contractions. This discovery (that alpha 1A and alpha 1D subtypes are only expressed in human bladder) together with the evidence that the alpha 1A subtype is expressed in prostatic smooth muscle mediating its contraction, suggest that the development of alpha 1A/alpha 1D selective antagonists would indeed relief both obstructive and irritating symptoms of BPH, a possible mechanism associated to tamsulosin or SL-89, 0591 (Michelotti et al., Pharmacol. Ther. 88: 281-309, 2000). However, up to now, molecules endowed with the selectivity such as ones of the present invention, were not available. Therefore, the search for new molecules continues for bypassing the difficulties and disadvantages of the ones currently available for the treatment of BPH and other disturbances of the same genesis in the lower urinary tract.

Safrole is the main constituent of the essential oil obtained from Sassafras or Cinnamon-Sassafras (Ocotea sp.), and is one of the main components of volatile oils from Brazil, occurring in different species of Brazilian cinnamon found in the South. It can be obtained with a purity greater than 95% and efficiencies around 80%, through simple distillation of the raw oil under reduced pressure. For these reasons, its use in the manufacture of bioactive substances presents advantages that include local availability, easy synthesis, and reduced costs. At the present invention safrole was used as the initial component for the derivatives synthesis that presented elevated alpha-adrenergic selective antagonist profile.

### BRIEF DESCRIPTION OF THE DRAWINGS

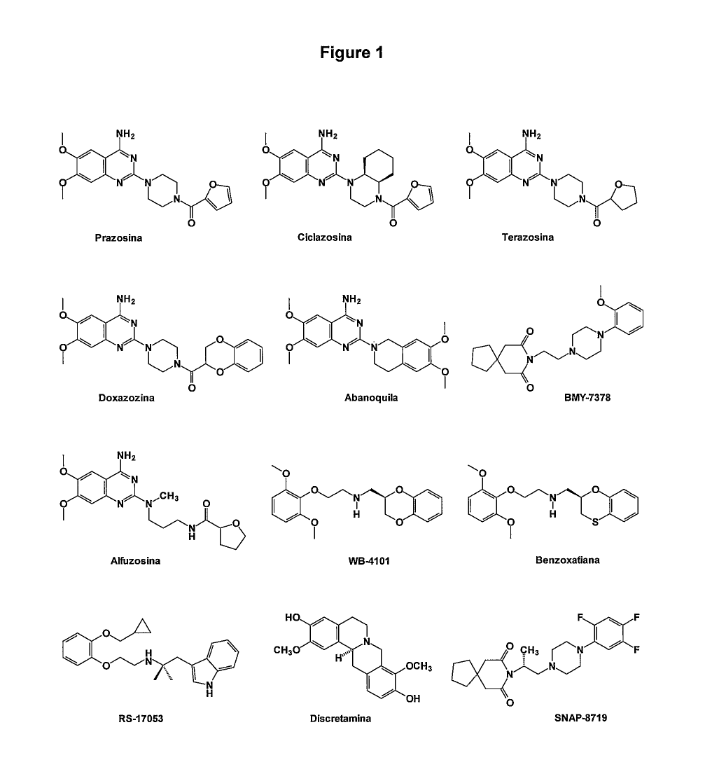

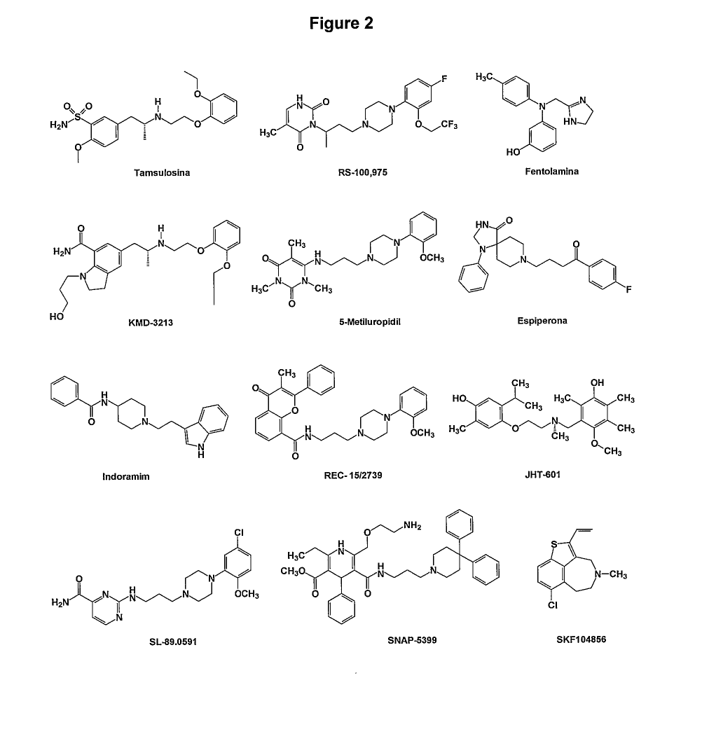

The FIGS. 1 and 2 show structures of synthetic derivatives described in the literature as alpha 1 adrenoceptors antagonists.

### SUMMARY OF THE INVENTION

It is an object of the present invention to provide options for currently available adrenergic antagonist molecules. In one aspect of the invention, the difficulties found with currently available adrenergic antagonist molecules are bypassed, since the present invention molecules are selective antagonists for the alpha 1A/alpha 1D receptors. It is therefore another object of the present invention to provide the use of new adrenergic selective antagonists for the alpha-1A/alpha 1D receptors and that, therefore, do not provoke the side effects associated with the use of the previously available molecules.

Another object of the present invention is to provide alpha 1A receptor antagonists, which are useful for the tissue relaxation of the lower urinary tract in mammals, preferentially humans.

Still is an object of the present invention to provide alpha 1D receptor antagonists, that are useful for the relief of irritating symptoms of bladder flow obstruction in mammals, preferentially humans.

It is, therefore, an additional object of the present invention to provide pharmaceutical compositions comprising new compounds for the treatment of lower urinary tract symptoms (LUTS).

The molecules of the present invention are particularly useful for benign prostatic hyperplasia (BPH) treatment. Therefore, an additional object of the present invention is to provide pharmaceutical compositions for the benign prostatic hyperplasia treatment. Given the advantages of the molecules' selectivity of the present invention, the pharmaceutical compositions of the present invention can be administrated in a larger variety of presentation forms, which results in benefits for the user and larger production flexibility. Therefore, another object of the present invention is to provide options for the limitations of administration of pharmaceutical compositions for benign prostatic hyperplasia treatment.

The present invention molecules have distinct synthesis pathways from other molecules currently used for BPH treatment. Moreover, the synthetic steps involved in the production of compounds of the present invention are simpler and less costly, resulting in advantages in the industrial point of view. Therefore, an additional object of the present invention is to provide methods for the production of adrenergic antagonists molecules described herein.

### DETAILED DESCRIPTION OF THE INVENTION

For this invention, “pharmaceutical compositions” is understood as all and any composition that contains an active ingredient, with prophylactic, palliative and/or curative ends, acting to maintain and/or to restore the homeostasis, can be topic, parenteral, enteral and/or intratecal administrated. “Active ingredient” is understood as all or any compound of formula (I) or (II), or pharmaceutically acceptable salts thereof. For this invention, the compound named LASSBIO 772 corresponds to the compound 1-(2-1,3-Benzodioxol-5-yl-ethyl)-4-(2-methoxy-phenyl)-piperazine and the compound named LASSBio 772B corresponds to the compound 4-Phenethyl-1-(2-methoxy-phenyl)-piperazine.

Despite some of the characteristic symptoms of the lower urinary tract, for instance, the irritation caused by obstruction and contraction of the urinary tract tissue, they do not have yet totally elucidated causes, being the benign prostatic hyperplasia one of many causes, this invention considers “lower urinary tract symptoms” (LUTS) symptoms as the irritation caused by obstruction, contraction of the urinary tract tissue and benign prostatic hyperplasia.

This invention has as one of the innovative characteristics the synthesis of phenylpiperazine derivatives of formula (I), rationally planned as selective antagonists for alpha 1A and alpha 1D adrenoceptors. These derivatives show as main structural characteristics the standard phenylpiperazine, where the tertiary amine function consists of pharmacophoric group involvement in the primary recognition by the alpha 1-adrenergic receptors; for the derivatives containing the 3,4-methylenedioxyphenyl subunit, this acts as secondary pharmacophoric group, with biophoric characteristics (electronic and hydrophobic) necessary for the recognition by (alpha) 1A-adrenoceptor, through electronic interactions of their oxygens with hydrogen bond donor sites, as well as Van der Waals interactions between its aromatic ring and similar sites. Studies accomplished by the inventors indicated that the absence of hydrogen-bonding acceptor (HBA) groups, in the secondary pharmacophoric subunit, does not influence the molecular recognition for (alpha) 1D-adrenoceptor, suggesting little relevance for the methylenedioxy subunit or the existence of specific complementary sites for electronic interactions and hydrophobic aromatics.

The utilization of this structural pattern for analogs of alpha 1-adrenergic antagonists has never been previously described and, therefore, the compound described in this invention and its synthetic methodology represent an innovation among alpha 1 antiadrenergic agents, although oxoanalogue derivatives, (X=O) n=2 and 3, have been claimed as anxiolytic agents (U.S. Pat. No. 5,219,855).

Additionally, the compounds of the present invention antagonize the alpha-1 receptors in nanomolar and sub-nanomolar concentrations, presenting affinity for alpha 1A and alpha 1D adrenoceptors, at least, a thousand times greater in comparison to (alpha) 1-B adrenoceptor. These concentration values are compatible with the requirements for the use of these molecules in pharmaceutical compositions.

The compounds of the present invention and their isotheres have general structure given by the formula (I) below:

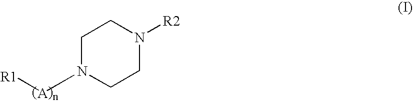

Where:

R1 corresponds to

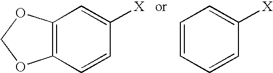

X corresponds to methylene, oxygen, sulfur or nitrogen;

A corresponds to CH₂ or CH(CH₃);

n represents an integer between 0 and 4;

R2 is hydrogen, alkyl, cycloalkyl; furyl, thiophenyl, pyridinyl, pyrimidinyl, pyrrolyl, thiazolyl, quinazolyl, isoquinolyl or phenyl-W;

where:

W is hydrogen, ortho-alkyl, ortho-cycloalkyl, ortho-alkoxyl, ortho-cycloalkoxyl, ortho-thioxyl, ortho-aryloxyl, ortho-sulfones, ortho-sulphide, ortho-sulfoxides, ortho-sulfonates, ortho-sulfonamides, ortho-amine, ortho-amide, ortho-halides, ortho-carboalkoxyl, ortho-carbothioalkoxyl, ortho-trihaloalkene, ortho-ciano, ortho-nitro, meta-alkyl, meta-cycloalkyl, meta-alkoxyl, meta-clycloalkoxyl, meta-thioxyl, meta-aryloxyl, meta-sulfones, meta-sulfetos, meta-sulfoxides, meta-sulfonates, meta-sulfonamides, meta-amine, meta-amide, meta-halides, meta-carboalkoxyl, meta-carbothioalkoxyl, meta-trihaloalkane, meta-ciano, meta-nitro, para-alkyl, para-cycloalkyl, para-alkoxyl, para-cycloalkoxyl, para-thioxyl, para-aryloxyl, para-sulfones, para-suphide, para-sulfoxides, para-sulfonates, para-sulfonamides, para-amine, para-amide, para-halides, para-carboalkoxyl, para-carbothioalkoxyl, para-trihaloalkene, para-cyano, para-nitro.

The compounds of formula (I) were obtained using synthetic methodology described herein, which is characterized by presenting few steps, with elevated efficiencies, starting from commercially available compounds, that qualifies this synthetic methodology for industrial utilization.

The compounds of the present invention were planned through convergent synthesis, using classical reactions, for example:
O-alkylation/reduction;
KNOEVENAGEL or DOEBNER condensation;
ozonolysis;
hydroboration followed by oxydative treatment;
homologation via cyamide followed by amidate hydrolysis via PINNER reaction;
reductive amination with aldehyde; and
amino-mercuration/reduction

More specifically, the compounds of formula (I), X=O (oxygen), of the present invention can be prepared by a method that comprises the steps of:

1. safrole isomeryzation,

2. ozonolysis,

3. BAYER-VILLIGER reaction or DAKIN oxydation;

4. O-alkylation with alpha-haloesthers

5. reduction;

6. condensation reaction with mesyl chloride (methylsulfonyl chloride);

7. bimolecular nucleophilic substitution reaction with substituted phenylpiperazine.

The step 2 reaction of the above method aims to prepare an aldehyde, such as, piperonal. The step 3 reaction aims to promote an oxydation in the molecule generating a phenol as product, such as, sesamol. The step 4 reaction aims to obtain beta-oxoesthers by the phenol O-alkylation. The step 5 reaction aims to obtain the corresponding alcohols by the beta-oxoesthers reduction. The step 6 reaction aims to obtain corresponding mesylates from the utilized alcohols. Additionally, this step is carried out in dichloromethane in the presence of triethanolamine. The step 7 reaction uses commercially available, free form substituted phenylpiperazin. Additionally, in this step is used as acetonitrile solvent in reflux or dimethylformamide (DMF) at 70° C. and a carbonate-alkaline base.

The compounds of formula (I), X=CH₂ (methylene), n=2, of the present invention can be prepared by a method that comprises the steps of:

1. safrole/reduction ozonolysis

2. condensation reaction with mesyl chloride (methylsulfonil chloride);

3. bimolecular nucleophilic substitution reaction with substituted phenylpiperazine.

The step 1 reaction of the above method aims to prepare an alcohol, such as, homo piperonyl or 3,4-methylenedioxyphenethyl. The step 2 reaction aims to obtain corresponding mesilate from the utilized alcohols. Additionally, this step carries out in dichloromethane in the presence of triethanolamine. The step 3 reaction uses commercially available free form substituted phenylpiperazine. Additionally, in this step is used as acetonitrile solvent in reflux or dimethylformamide (DMF) at 70° C. and a carbonate-alkaline base.

The compounds of formula (I), X=CH₂ (methylene), n=3, of the present invention can be prepared by a method that comprises the steps of:

1. KNOEVENAGEL or DOEBNER condensation;

2. reduction

3. condensation reaction with mesyl chloride (methylsulfonil chloride);

4. bimolecular nucleophilic substitution reaction with substituted phenylpiperazine.

The step 1 reaction of the above method aims to prepare an alfa,beta-unsaturated acid, such as, 3,4-methyleneodioxycinnamic. The step 2 reaction aims to obtain the saturated alcohol, such as the 3,4-methyleneodioxycinnamyl. The step 3 reaction aims to obtain corresponding mesilates from the utilized alcohols. Additionally, this step carries out in dichloromethane in the presence of triethanolamine. The step 4 reaction uses commercially available free form substituted phenylpiperazine. Additionally, in this step is used as acetonitrile solvent in reflux or dimethylformamide (DMF) at 70° C. and a carbonate-alkaline base.

The compounds of formula (I), X=CH₂ (methylene), n=3, of the present invention can be prepared by a method that comprises the steps of:

1. safrole hydroboration;

2. condensation reaction with mesyl chloride (methylsulfonil chloride);

3. bimolecular nucleophilic substitution reaction with substituted phenylpiperazine.

The step 1 reaction of the above method aims to prepare a saturated alcohol, such as 3,4-methylenedioxycinnamyl. The step 2 reaction aims to obtain corresponding mesilates from the utilized alcohols. Additionally, this step carries out in dichloromethane in the presence of triethanolamine. The step 3 reaction uses commercially available free form substituted phenylpiperazine. Additionally, in this step is used as acetonitrile solvent in reflux or dimethylformamide (DMF) at 70° C. and a carbonate-alkaline base.

The compounds of formula (I), X=CH₂ (methylene), n=4, of the present invention can be prepared by a method that comprises the steps of:

1. homologation via cyamide followed by amidate hydrolysis via PINNER Reaction;

2. reduction;

3. condensation reaction with mesyl chloride (methylsulfonil chloride);

4. bimolecular nucleophilic substitution reaction with substituted phenylpiperazine.

The step 1 reaction of the above method aims to prepare an esther, such as, methyl 4-(3,4-methyleneodioxyphenyl)butyrate. The step 2 reaction aims obtain the corresponding alcohol, such as 4-(3,4-methyleneodioxyphenyl)butyl. The step 3 reaction aims to obtain corresponding mesilates from the utilized alcohols. Additionally, this step carries out in dichloromethane in the presence of triethanolamine. The step 4 reaction uses commercially available free form substituted phenylpiperazine. Additionally, in this step is used as acetonitrile solvent in reflux or dimethylformamide (DMF) at 70° C. and a carbonate-alkaline base.

The compounds of formula (I), of the present invention can also be prepared by a method that comprises the steps:

1. safrole amine-mercuration;

2. reduction with sodium borohydride.

The step 1 of the method above uses mercury acetate in THF/water.

For exemplification, we describe in this report the synthesis of the following compounds:
1-benzo[d][1,3]dioxol-5-yloxy-2-(4-phenylhexahydro-1-pyrazinyl)ethane 1-benzo[d][1,3]dioxol-5-yloxy-2-[4-(4-fluorophenyl)hexahydro-1-pyrazinyl]ethane
1-benzo[d][1,3]dioxol-5-yloxy-2-[4-(4-chlorophenyl)hexahydro-1-pyrazinyl]propane
1-benzo[d][1,3]dioxol-5-yloxy-2-[4-(4-methoxyphenyl)hexahydro-1-pyrazinyl]ethane
1-benzo[d][1,3]dioxol-5-yloxy-2-[4-(2-pirimidinil)hexahydro-1-pyrazinyl]ethane
1-benzo[d][1,3]dioxol-5-yloxy-3-(4-phenylhexahydro-1-pyrazinyl)propane
1-benzo[d][1,3]dioxol-5-yloxy-3-[4-(4-fluorophenyl)hexahydro-1-pyrazinyl]propane
1-benzo[d][1,3]dioxol-5-yloxy-3-[4-(4-chlorophenyl)hexahydro-1-pyrazinyl]propane
1-benzo[d][1,3]dioxol-5-yloxy-3-[4-(4-methoxyphenyl)hexahydro-1-pyrazinyl]propane
1-benzo[d][1,3]dioxol-5-yloxy-3-[4-(2-pirimidinil)hexahydro-1-pyrazinyl]propane
1-benzo[d][1,3]dioxol-5-yl-2-(4-phenylhexahydro-1-pyrazinyl)ethane 1-benzo[d][1,3]dioxol-5-yl-2-[4-(4-fluorophenyl)hexahydro-1-pyrazinyl]ethane
1-benzo[d][1,3]dioxol-5-yl-2-[4-(4-chlorophenyl)hexahydro-1-pyrazinyl]ethane
1-benzo[d][1,3]dioxol-5-yl-2-[4-(2-methoxyphenyl)hexahydro-1-pyrazinyl]ethane
1-benzo[d][1,3]dioxol-5-yl-2-[4-(4-methoxyphenyl)hexahydro-1-pyrazinyl]ethane
1-benzo[d][1,3]dioxol-5-yl-3-(4-phenylhexahydro-1-pyrazinyl)propane 1-benzo[d][1,3]dioxol-5-yl-3-[4-(4-fluorophenyl)hexahydro-1-pyrazinyl]propane
1-benzo[d][1,3]dioxol-5-yl-3-[4-(4-chlorophenyl)hexahydro-1-pyrazinyl]propane
1-benzo[d][1,3]dioxol-5-yl-3-[4-(2-methoxyphenyl)hexahydro-1-pyrazinyl]propane
1-benzo[d][1,3]dioxol-5-yl-3-[4-(4-methoxyphenyl)hexahydro-1-pyrazinyl]propane
1-benzo[d][1,3]dioxol-5-yl-3-[4-(2-pirimidinil)hexahydro-1-pyrazinyl]propane
1-benzo[d][1,3]dioxol-5-yl-4-(4-phenylhexahydro-1-pyrazinyl)butane
1-benzo[d][1,3]dioxol-5-yl-4-[4-(4-fluorophenyl)hexahydro-1-pyrazinyl]butane
1-benzo[d][1,3]dioxol-5-yl-4-[4-(4-chlorophenyl)hexahydro-1-pyrazinyl]butane
1-benzo[d][1,3]dioxol-5-yl-4-[4-(4-methoxyphenyl)hexahydro-1-pyrazinyl]butane
1-benzo[d][1,3]dioxol-5-yl-4-[4-(2-pirimidinil)hexahydro-1-pyrazinyl]butane

A detailed description of the synthetic methods of this invention for some of the claimed compounds is described as follows, including important spectroscopic data for their characterization. The next examples illustrate, but do not limit the present invention.

### EXAMPLE 1

### Preparation of 1-benzo[d][1,3]dioxol-5-yloxy-2-[4(4-W-substituted-phenyl)hexahydro-1-pyrazinyl]ethane Derivatives

General Procedure

In a 15 mL balloon containing a mixture of 0.130 g of 2-(3,4-methyleneodioxyphenyl)-1-yloxy-ethaneyl mesilate (0.50 mMol), 0.074 g of lithium carbonate (1.00 mMol) in acetonitrile (6 mL), was added 2.00 mMol of phenylpiperazine. The mixture was kept in reflux for 24 hours under vigorous agitation and nitrogen atmosphere. At the end of this time the solution was concentrated in a rotatory evaporator, solubilized in dichloromethane and mixed in silica gel. The material was chromatografied in a silica gel column eluted with dichloromethane, followed by chloroform supplying the desired compounds.

### 1-benzo[d][1,3]dioxol-5-yloxy-2-(4-phenylhexahydro-1-pyrazinyl)ethane

Solid white, 0.154 g (94%), Rf=0.58 (CHCl₃:EtOH 5%), p.f.: 95-96° C.

RMN ¹H (200 MHz, CDCl₃): δ 2.72 (m, 4H, ArNCH₂CH₂N); 2.82 (t, J=5.59 Hz, 2H, OCH₂CH₂N); 3.21(m, 4H, ArNCH₂CH₂N); 4.05 (t, J=5.59 Hz, 2H, OCH₂CH₂N); 5.89 (s, 2H, OCH₂O); 6.33 (dd, J²=8.61 Hz, J³=1.93, 1H, Ar—H-6′); 6.51 (d, J³=1.93 Hz 1H, Ar—H-2′); 6.69 (d, J²=8.61 Hz, 1H, Ar—H-5′); 6.82 (m, N—Ar—H-4″); 6.92 (m, N—Ar—H-2″); 7.25 (m, N—Ar—H-3″);

RMN ¹³C (50 MHz, CDCl₃): δ 48.9 (ArNCH₂CH₂N); 53.4 (NCH₂CH₂O); 57.0 (ArNCH₂CH₂N); 66.7 (NCH₂CH₂O); 98.0 (Ar-2′-CH); 100.9 (OCH₂O); 105.6 (Ar-5′-CH); 107.7 (Ar-6′-CH); 115.8 (Ar-2″ e 6″-2CH); 119.5 (Ar-4″-CH); 128.9 (Ar-3″ e 5″-2CH); 141.5 (Ar-4′-C); 148.0 (Ar-3′-C); 151.0 (Ar-1″-C—N); 154.0 (Ar-1′-C—O).

### 1-benzo[d][1,3]dioxol-5-yloxy-2-[4-(4-fluorophenyl)hexahydro-1-pyrazinyl]ethane

Solid white 0.158 g (92%), Rf=0.50 (CHCl₃:EtOH 5%), p.f.: 95-96° C.

RMN ¹H (200 MHz, CDCl₃): δ 2.74 (m, 4H, ArNCH₂CH₂N); 2.83 (t, J=5.59 Hz, 2H, OCH₂CH₂N); 3.15 (m, 4H, ArNCH₂CH₂N); 4.06 (t, J=5.59 Hz, 2H, OCH₂CH₂N); 5.90 (s, 2H, OCH₂O); 6.34 (dd, J²=8.42 Hz, J³=2.20, 1H, Ar—H-6′); 6.52 (d, J³=2.20 Hz 1H, Ar—H-2′); 6.70 (d, J²=8.42 Hz, 1H, Ar—H-5′); 6.87 (m, 2H, N—Ar—H-2″ e 6″); 6.96 (m, 2H, N—Ar—H-3″ e 5″).

RMN ¹³C (50 MHz, CDCl₃): δ 49.9 (ArNCH₂CH₂N); 53.4 (NCH₂CH₂O); 57.0 (ArNCH₂CH₂N); 66.7 (NCH₂CH₂O); 98.1 (Ar-2′-CH); 100.9 (OCH₂O); 105.6 (Ar-5′-CH); 107.7 (Ar-6′-CH); 115.3 (Ar-3″ e 5″-2CH); 117.6 (Ar-2″ e 6″-2CH); 141.5 (Ar-4′-C); 147.8 (Ar-1″-C—N); 148.0 (Ar-3′-C); 154.0 (Ar-1′-C—O) 154.6 (Ar-4″-C—F); 159.3 (Ar-4″-C—F).

### 1-benzo[d][1,3]dioxol-5-yloxy-2-[4-(4-chlorophenyl)hexahydro-1-pyrazinyl]ethane

Solid white, 0.168 g (93%), Rf=0.54 (CHCl₃:EtOH 5%),p.f.: 106-107° C.

RMN ¹H (200 MHz, CDCl₃): δ 2.71 (m, 4H, ArNCH₂CH₂N); 2.82 (t, J=5.58 Hz, 2H, OCH₂CH₂N); 3.17 (m, 4H, ArNCH₂CH₂N); 4.05 (t, J=5.58 Hz, 2H, OCH₂CH₂N); 5.90 (s, 2H, OCH₂O); 6.33 (dd, J²=8.42 Hz, J³=2.29, 1H, Ar—H-6′); 6.51 (d, J³=2.29 Hz 1H, Ar—H-2′); 6.69 (d, J²=8.42 Hz, 1H, Ar—H-5′); 6.82 (d, J²=8.79 Hz, 2H, N—Ar—H-2″ e 6″); 7.19 (d, J²=8.79, 2H, N—Ar—H-3″ e 5″).

RMN ¹³C (50 MHz, CDCl₃): δ 48.9 (ArNCH₂CH₂N); 53.3 (NCH₂CH₂O); 57.0 (ArNCH₂CH₂N); 66.7 (NCH₂CH₂O); 98.1 (Ar-2′-CH); 100.9 (OCH₂O); 105.6 (Ar-5′-CH); 107.7 (Ar-6′-CH); 117.0 (Ar-2″ e 6″-2CH); 124.3 (Ar-4″-C-Cl); 128.7 (Ar-3″ e 5″-2CH); 141.6 (Ar-4′-C); 148.0 (Ar-3′-C); 149.0 (Ar-1″-C—N); 154.0 (Ar-1′-C—O).

### 1-benzo[d][1,3]dioxol-5-yloxy-2-[4-(4-methoxyphenyl)hexahydro-1-pyrazinyl]ethane

Solid white, 0.164 g (92%), Rf=0.48 (CHCl₃:EtOH 5%), p.f.: 116-117° C.

RMN ¹H (200 MHz, CDCl₃): δ 2.73 (m, 4H, ArNCH₂CH₂N); 2.82 (t, J=5.77 Hz, 2H, OCH₂CH₂N); 3.11 (m, 4H, ArNCH₂CH₂N); 3.75 (s, 3H, OCH₃); 4.05 (t, J=5.77 Hz, 2H, OCH₂CH₂N); 5.90 (s, 2H, OCH₂O); 6.33 (dd, J²=8.42 Hz, J³=2.47, 1H, Ar—H-6′); 6.51 (d, J³=2.47 Hz, 1H, Ar—H-2′); 6.69 (d, J²=8.42 Hz, 1H, Ar—H-5′); 6.81 (d, 2H, N—Ar—H-2″ e 6″); 6.89 (d, 2H, N—Ar—H-3″ e 5″).

RMN ¹³C (50 MHz, CDCl₃): δ 50.4 (ArNCH₂CH₂N); 53.6 (NCH₂CH₂O); 55.4 (Ar-4″-OCH₃); 57.1 (ArNCH₂CH₂N); 67.0 (NCH₂CH₂O); 98.2 (Ar-2′-CH); 100.9 (OCH₂O); 105.8 (Ar-5′-CH); 107.7 (Ar-6′-CH); 114.0 (Ar-3″ e 5″-2CH); 118.0 (Ar-2″ e 6″-2CH); 141.6 (Ar-4′-C); 145.6 (Ar-1″-C—N); 148.1 (Ar-3′-C); 153.7 (Ar-1′-C—O); 154.2 (Ar-4″-C—OCH₃).

### 1-benzo[d][1,3]dioxol-5-yloxy-2-[4-(2-pyrimidinyl)hexahydro-1-pyrazinyl]ethane

Solid white 0.152 g (93%), Rf=0.36 (CHCl₃:EtOH 5%), p.f.: 69-71° C.

RMN ¹H (200 MHz, CDCl₃): δ 2.61 (m, 4H, ArNCH₂CH₂N); 2.80 (t, J=5.76 Hz, 2H, OCH₂CH₂N); 3.85 (m, 4H, ArNCH₂CH₂N); 4.06 (t, J=5.76 Hz, 2H, OCH₂CH₂N); 5.90 (s, 2H, OCH₂O); 6.33 (dd, J²=8.42 Hz, J³=2.20, 1H, Ar—H-6′); 6.46 (dd, J²=4.72 Hz, 1H, Ar—H-4″); 6.51 (d, J³=2.20 Hz, 1H, Ar—H-2′); 6.69 (dd, J²=8.42 Hz, 1H, Ar—H-5′); 8.29 (dd, j 2=8.42 Hz, J³=0.40 Hz, 2H, N—Ar—H-3″ e 5″);

RMN ¹³C (50 MHz, CDCl₃): δ 43.5 (ArNCH₂CH₂N); 53.3 (NCH₂CH₂O); 57.2 (ArNCH₂CH₂N); 66.7 (NCH₂CH₂O); 98.1 (Ar-2′-CH); 100.9 (OCH₂O); 105.7 (Ar-5′-CH); 107.7 (Ar-6′-CH); 109.6 (Ar-4″-CH); 141.6 (Ar-4′-C); 148.0 (Ar-3′-C); 154.1 (Ar-1′-C—O); 157.5 (Ar-3″ e 5″-2CH); 161.5 (Ar-1″-C—N).

### EXAMPLE 2

### Preparation of 1-benzo[d][1,3]dioxol-5-yloxy-3-[4(4-W-substituted-phenyl)hexahydro-1-pyrazinyl]propane Derivatives

General Procedure

In a 15 mL balloon containing a mixture of 0.104 g of 2-(3,4-methylenedioxophenyl)-1-yloxy-propanyl mesilate (0.38 mMol), 0.074 g of lithium carbonate (1.00 mMol) in acetonitrile (6 mL), was added 1.05 mMol of phenylpiperazine. The mixture was kept in reflux for 24 hours under vigorous agitation and nitrogen atmosphere. At the end of this time the solution was concentrated in a rotatory evaporator, solubilized in dichloromethane and mixed in a silica gel. The material was chromatografied in a silica gel column eluted with dichloromethane, followed by chloroform supplying the desired compound.

### 1-benzo[d][1,3]dioxol-5-yloxy-3-(4-phenylhexahydro-1-pyrazinyl)propane

solid light beige, 0.121 g (92%), Rf=0.45 (CHCl₃:EtOH 5%), p.f.: 80-82° C.

RMN ¹H (200 MHz, CDCl₃): δ 1.96 (qi, J=6.13, 2H, OCH₂CH₂CH₂N); 2.55 (t, J=6.13, 2H, OCH₂CH₂CH₂N); 2.61 (m, 4H, ArNCH₂CH₂N); 3.21 (m, 4H, ArNCH₂CH₂N); 3.95 (t, J=6.13 Hz, 2H, OCH₂CH₂CH₂N); 5.88 (s, 2H, OCH₂O); 6.32 (dd, J²=8.43 Hz, J³=1.92, 1H, Ar—H-6′); 6.50 (d, J³=1.92 Hz 1H, Ar—H-2′); 6.69 (d, J²=8.43 Hz, 1H, Ar—H-5′); 6.84 (m, N—Ar—H-4″); 6.94 (m, N—Ar—H-2″ e 6″); 7.25 (m, N—Ar—H-3″ e 5″);

RMN ¹³C (50 MHz, CDCl₃): δ 26.6 (NCH₂CH₂CH₂O) 48.9 (ArNCH₂CH₂N); 53.1 (ArNCH₂CH₂N); 55.0 (NCH₂CH₂CH₂O); 67.0 (NCH₂CH₂CH₂O); 97.9 (Ar-2′-CH); 100.9 (OCH₂O); 105.5 (Ar-5′-CH); 107.7 (Ar-6′-CH); 115.8 (Ar-2″ e 6″-2CH); 119.5 (Ar-4″-CH); 128.9 (Ar-3″ e 5″-2CH); 141.3 (Ar-4′-C); 148.0 (Ar-3′-C); 151.1 (Ar-1″-C—N); 154.0 (Ar-1′-C—O).

### 1-benzo[d][1,3]dioxol-5-yloxy-3-[4-(4-fluorophenyl)hexahydro-1-pyrazinyl]propane

Solid white, 0.131 g (95%), Rf=0.42 (CHCl₃:EtOH 5%), p.f.: 80-82° C.

RMN ¹H (200 MHz, CDCl₃): δ 1.96 (qi, J=7.42, 2H, OCH₂CH₂CH₂N); 2.57 (t, J=7.42, 2H, OCH₂CH₂CH₂N); 2.62 (m, 4H, ArNCH₂CH₂N); 3.12 (m, 4H, ArNCH₂CH₂N); 3.95 (t, J=6.25 Hz, 2H, OCH₂CH₂CH₂N); 5.89 (s, 2H, OCH₂O); 6.32 (dd, J²=8.43 Hz, J³=2.45, 1H, Ar—H-6′); 6.50 (d, J³=2.45 Hz 1H, Ar—H-2′); 6.69 (d, J²=8.43 Hz, 1H, Ar—H-5′); 6.91 (m, 5H, N—Ar—H-2″, 3″, 5″ e 6″);

RMN ¹³C (50 MHz, CDCl₃): δ 26.6 (NCH₂CH₂CH₂O) 49.9 (ArNCH₂CH₂N); 53.1 (ArNCH₂CH₂N); 54.9 (NCH₂CH₂CH₂O); 66.9 (NCH₂CH₂CH₂O); 97.9 (Ar-2′-CH); 100.9 (OCH₂O); 105.5 (Ar-5′-CH); 107.7 (Ar-6′-CH); 115.8 (Ar-3″ e 5″-2CH); 117.5 (Ar-2″ e 6″-2CH); 141.4 (Ar-4′-C); 147.8 (Ar-1″-C—N); 148.0 (Ar-3′-C); 154.3 (Ar-1′-C—O) 154.6 (Ar-4″-C—F); 159.3 (Ar-4″-C—F).

### 1-benzo[d][1,3]dioxol-5-yloxy-3-[4-(4-chlorophenyl)hexahydro-1-pyrazinyl]propane

Solid white 0.136 g (94%), Rf=0.52 (CHCl₃:EtOH 5%), p.f.: 89-90° C.

RMN ¹H (200 MHz, CDCl₃): δ 1.95 (qi, J=6.50, 2H, OCH₂CH₂CH₂N); 2.55 (t, J=6.50, 2H, OCH₂CH₂CH₂N); 2.60 (m, 4H, ArNCH₂CH₂N); 3.16 (m, 4H, ArNCH₂CH₂N); 3.95 (t, J=6.13 Hz, 2H, OCH₂CH₂CH₂N); 5.90 (s, 2H, OCH₂O); 6.31 (dd, J²=8.42 Hz, J³=2.47, 1H, Ar—H-6′); 6.48 (d, J³=2.47 Hz 1H, Ar—H-2′); 6.68 (d, J²=8.42 Hz, 1H, Ar—H-5′); 6.83 (m, 2H, N—Ar—H-2″ e 6″); 7.18 (m, 2H, N—Ar—H-3″ e 5″);

RMN ¹³C (50 MHz, CDCl₃): δ 26.6 (NCH₂CH₂CH₂O) 48.9 (ArNCH₂CH₂N); 52.9 (ArNCH₂CH₂CH₂N); 54.9 (NCH₂CH₂CH₂O); 66.9 (NCH₂CH₂O); 97.9 (Ar-2′-CH); 100.9 (OCH₂O); 105.5 (Ar-5′-CH); 107.7 (Ar-6′-CH); 116.9 (Ar-2″ e 6″-2CH); 124.2 (Ar-4″-C-Cl); 128.7 (Ar-3″ e 5″-2CH); 141.3 (Ar-4′-C); 148.0 (Ar-3′-C); 149.7 (Ar-1″-C—N); 154.3 (Ar-1′-C—O).

### 1-benzo[d][1,3]dioxol-5-yloxy-3-[4-(4-methoxyphenyl)hexahydro-1-pyrazinyl]propane

Solid white, 0.131 (92%), Rf=0.46 (CHCl₃:EtOH 5%), p.f.: 104-106° C.

RMN ¹H (200 MHz, CDCl₃): δ 1.95 (qi, J=6.50, 2H, OCH₂CH₂CH₂N); 2.55 (t, J=6.50, 2H, OCH₂CH₂CH₂N); 2.62 (m, 4H, ArNCH₂CH₂N); 3.09 (m, 4H, ArNCH₂CH₂N); 3.75 (s, 3H, OCH₃); 3.95 (t, J=6.50 Hz, 2H, OCH₂CH₂CH₂N); 5.90 (s, 2H, OCH₂O); 6.31 (dd, J²=8.42 Hz, J³=2.47, 1H, Ar—H-6′); 6.49 (d, J³=2.47 Hz 1H, Ar—H-2′); 6.68 (d, J²=8.42 Hz, 1H, Ar—H-5′); 6.82 (m, 2H, N—Ar—H-2″ e 6″); 6.89 (m, 2H, N—Ar—H-3″ e 5″);

RMN ¹³C (50 MHz, CDCl₃): δ 26.7 (NCH₂CH₂CH₂O) 50.4 (ArNCH₂CH₂N); 53.2 (ArNCH₂CH₂N); 55.0 (NCH₂CH₂CH₂O); 55.4 (Ar-4″-C—OCH₃); 67.1 (NCH₂CH₂CH₂O); 97.9 (Ar-2′-CH); 100.9 (OCH₂O); 105.6 (Ar-5′-CH); 107.7 (Ar-6′-CH); 114.3 (Ar-3″ e 5″-2CH); 117.9 (Ar-2″ e 6″-2CH); 141.3 (Ar-4′-C); 145.6 (Ar-1″-C—N); 148.0 (Ar-3′-C); 153.6 (Ar-1-C—O); 154.4 (Ar-4″-C—OCH₃).

### 1-benzo[d][1,3]dioxol-5-yloxy-3-[4-(2-pyrimidinyl)hexahydro-1-pyrazinyl]propane

Yellowish liquid, 0.130 g (98%), Rf=0.48 (CHCl₃:EtOH 5%).

Molecular formula: C₁₈H₂₂N₄O₃

RMN ¹H (200 MHz, CDCl₃): δ 1.96 (qi, J=6.50, 2H, OCH₂CH₂CH₂N); 2.57 (t, J=6.50, 2H, OCH₂CH₂CH₂N); 2.61 (m, 4H, ArNCH₂CH₂N); 3.09 (m, 4H, ArNCH₂CH₂N); 3.75 (s, 3H, OCH₃); 3.95 (t, J=6.50 Hz, 2H, OCH₂CH₂CH₂N); 5.89 (s, 2H, OCH₂O); 6.32 (dd, J²=8.42 Hz, J³=2.20, 1H, Ar—H-6′); 6.50 (d, J³=2.20 Hz 1H, Ar—H-2′); 6.46 (m, 1H, N—Ar—H-4″); 6.69 (d, J²=8.42 Hz, 1H, Ar—H-5′); 8.29 (m, 2H, N—Ar—H-3″ e 5″).

RMN ¹³C (50 MHz, CDCl₃): δ 26.6 (NCH₂CH₂CH₂O) 43.5 (ArNCH₂CH₂N); 57.2 (ArNCH₂CH₂N); 53.3 (NCH₂CH₂CH₂O); 66.7 (NCH₂CH₂CH₂O); 98.1 (Ar-2′-CH); 100.9 (OCH₂O); 105.7 (Ar-5′-CH); 107.7 (Ar-6′-CH); 109.4 (Ar-4″-CH) 141.6 (Ar-4′-C); 148.0 (Ar-3′-C); 154.1 (Ar-1-C—O); 157.5 (Ar-3″ e 5″-2CH); 161.5 (Ar-1″-C—N).

### EXAMPLE 3

### Preparation of 1-benzo[d][1,3]dioxol-5-yl-2-[4(4-W-substituted-phenyl)hexahydro-1-pyrazinyl]ethane Derivatives

General Procedure

In a 15 mL balloon containing a mixture of 0.103 g of 2-(3,4-methylenedioxophenyl)-1-yl-ethanyl mesilate (0.42 mMol), 0.074 g of lithium carbonate (1.00 mMol) in acetonitrile (6 mL), was added 1.20 mMol of phenylpiperazine. The mixture was kept in reflux for 24 hours under vigorous agitation and nitrogen atmosphere. At the end of this time the solution was concentrated in rotatory evaporator, solubilized in dichloromethane and mixed in a silica gel. The material was chromatografied in a silica gel column eluted with dichloromethane, followed by chloroform supplying the desired compound.

### 1-benzo[d][1,3]dioxol-5-yl-2-(4-phenylhexahydro-1-pyrazinyl)ethane

Solid white 0.112 g (92%), Rf=0.57 (CHCl₃:EtOH 5%), p.f.: 85-86° C.

RMN ¹H (200 MHz, CDCl₃): δ 2.71 (m, 8H, ArCH₂CH₂N (4H) e ArNCH₂CH₂N (4H)) 3.17 (m, 4H, ArNCH₂CH₂N); 5.91 (s, 2H, OCH₂O); 6.65 (d, J²=7.70 Hz, J³=1.84, 1H, Ar—H-6′); 6.70 (m, J³=1.84, 1H, Ar—H-2′); 6.75 (d, J²=7.70 Hz, 1H, Ar—H-5′); 6.82 (m, N—Ar—H-4″); 6.92 (m, N—Ar—H-2″ e 6″); 7.25 (m, N—Ar—H-3″ e 5″).

RMN ¹³C (50 MHz, CDCl₃): δ 32.1 (NCH₂CH₂Ar); 48.9 (ArNCH₂CH₂N); 53.0 (ArNCH₂CH₂N); 60.4 (NCH₂CH₂Ar); 100.6 (OCH₂O); 106.0 (Ar-2′-CH); 107.7 (Ar-5′-CH); 115.8 (Ar-2″ e 6″-2CH); 119.5 (Ar-4″-CH); 128.9 (Ar-3″ e 5″-2CH); 121.3 (Ar-6′-CH); 128.8 (Ar-3″ e 5″-2CH); 135.6 (Ar-1′-C); 145.3 (Ar-4′-C); 147.3 (Ar-3′-C); 151.0 (Ar-1″-C—N).

### 1-benzo[d][1,3]dioxol-5-yl-2-[4-(4-fluorophenyl)hexahydro-1-pyrazinyl]ethane

Solid white, 0.124 g (90%), Rf=0.48 (CHCl₃:EtOH 5%), p.f.: 89-90° C.

RMN ¹H (200 MHz, CDCl₃): δ 2.70 (m, 8H, ArCH₂CH₂N (4H) e ArNCH₂CH₂N (4H)) 3.16 (m, 4H, ArNCH₂CH₂N); 5.91 (s, 2H, OCH₂O); 6.67 (d, J²=7.87 Hz, J³=1.83, 1H, Ar—H-6′); 6.73 (bd, 1H, Ar—H-2′); 6.76 (bd, 1H, Ar—H-5′); 6.88 (m, 2H, N—Ar—H-2″, e 6″); 6.98 (m, 2H, N—Ar—H-3″ e 5″).

RMN ¹³C (50 MHz, CDCl₃): δ 33.1 (NCH₂CH₂Ar); 48.9 (ArNCH₂CH₂N); 53.0 (ArNCH₂CH₂N); 60.4 (NCH₂CH₂Ar); 100.6 (OCH₂O); 108.0 (Ar-2′-CH); 109.0 (Ar-5′-CH); 115.3 (Ar-3″ e 5″-2CH); 117.7 (Ar-2″ e 6″-2CH); 121.3 (Ar-6′-CH); 133.7 (Ar-1′-C); 145.7 (Ar-4′-C); 147.4 (Ar-3′-C); 147.8 (Ar-1″-C—N); 156.6 (Ar-4″-C—F); 159.4 (Ar-4″-C—F).

### 1-benzo[d][1,3]dioxol-5-yl-2-[4-(4-chlorophenyl)hexahydro-1-pyrazinyl]ethane

Solid white, 0.132 (91%), Rf=0.50 (CHCl₃:EtOH 5%), p.f.: 94-95° C.

RMN ¹H (200 MHz, CDCl₃): δ 2.67 (m, 8H, ArCH₂CH₂N (4H) e ArNCH₂CH₂N (4H)) 3.17 (m, 4H, ArNCH₂CH₂N); 5.91 (s, 2H, OCH₂O); 6.65 (d, J²=7.69 Hz, J³=1.83, 1H, Ar—H-6′); 6.70 (m, J³=1.83, 1H, Ar—H-2′); 6.75 (d, J²=7.69 Hz, 1H, Ar—H-5′); 6.82 (m, 2H, N—Ar—H-2″, e 6″); 7.19 (m, 2H, N—Ar—H-3″ e 5″).

RMN ¹³C (50 MHz, CDCl₃): δ 33.1 (NCH₂CH₂Ar); 48.9 (ArNCH₂CH₂N); 52.9 (ArNCH₂CH₂N); 60.4 (NCH₂CH₂Ar); 100.6 (OCH₂O); 108.0 (Ar-2′-CH); 108.7 (Ar-5′-CH); 117.0 (Ar-2″ e 6″-2CH); 121.2 (Ar-6′-CH); 124.2 (Ar-4″-C-Cl); 128.7 (Ar-3″ e 5″-2CH); 133.7 (Ar-1′-C); 145.6 (Ar-4′-C); 147.4 (Ar-3′-C); 149.7 (Ar-1″-C—N).

### 1-benzo[d][1,3]dioxol-5-yl-2-[4-(2-methoxyphenyl)hexahydro-1-pyrazinyl]ethane

Solid white, 0.140 g (98%), Rf=0.55 (CHCl₃:EtOH 5%), p.f.: 90-92° C.

RMN ¹H (200 MHz, CDCl₃): δ 2.71 (m, 8H, ArCH₂CH₂N (4H) e ArNCH₂CH₂N (4H)) 3.14 (m, 4H, ArNCH₂CH₂N); 3.75 (s, 3H, OCH₃); 5.91 (s, 2H, OCH₂O); 6.65 (d, J²=7.88 Hz, J³=2.02, 1H, Ar—H-6′); 6.68 (m, J³=2.02, 1H, Ar—H-2′); 6.75 (d, J²=7.88 Hz, 1H, Ar—H-5′); 6.86 (d, J²=7.51 Hz, 1H, N—Ar—H-6″); 6.98 (m, 3H, N—Ar—H-3″, 4″ e 5″).

RMN ¹³C (50 MHz, CDCl₃): δ 33.1 (NCH₂CH₂Ar); 50.4 (ArNCH₂CH₂N); 53.2 (ArNCH₂CH₂N); 55.2 (Ar-2″-C—OCH₃); 60.6 (NCH₂CH₂Ar); 100.6 (OCH₂O); 108.0 (Ar-2′-CH); 109.0 (Ar-5′-CH); 111.0 (Ar-3″-CH) 118.0 (Ar-6″-CH); 120.8 (Ar-5″-CH) 121.2 (Ar-6′-CH); 122.7 (Ar-4″-CH); 133.9 (Ar-1′-C); 141.1(Ar-1″-C—N); 145.6 (Ar-4′-C) 147.3 (Ar-3′-C); 152.1 (Ar-2″-C—OCH₃).

### 1-benzo[d][1,3]dioxol-5-yl-2-[4-(4-methoxyphenyl)hexahydro-1-pyrazinyl]ethane

Solid white, 0.126 g (88%), Rf=0.44 (CHCl₃:EtOH 5%), p.f.: 112-113° C.

RMN ¹H (200 MHz, CDCl₃): δ 2.71 (m, 8H, ArCH₂CH₂N (4H) e ArNCH₂CH₂N (4H)) 3.14 (m, 4H, ArNCH₂CH₂N); 3.75 (s, 3H, OCH₃); 5.91 (s, 2H, OCH₂O); 6.65 (d, J²=7.88 Hz, J³=2.02, 1H, Ar—H-6′); 6.68 (m, J³=2.02, 1H, Ar—H-2′); 6.75 (d, J²=7.88 Hz, 1H, Ar—H-5′); 6.84 (m, 2H, N—Ar—H-2″, e 6″); 6.92 (m, 2H, N—Ar—H-3″ e 5″).

RMN ¹³C (50 MHz, CDCl₃): δ 33.1 (NCH₂CH₂Ar); 50.4 (ArNCH₂CH₂N); 53.1 (ArNCH₂CH₂N); 55.3 (Ar-4″-C—OCH₃); 60.4 (NCH₂CH₂Ar); 100.6 (OCH₂O); 108.0 (Ar-2′-CH); 108.9 (Ar-5′-CH); 114.2 (Ar-3″ e 5″-2CH); 117.9 (Ar-2″ e 6″-2CH); 121.2 (Ar-6′-CH); 133.8 (Ar-1′-C); 145.5 (Ar-4′-C e Ar-1″-C—N); 147.3 (Ar-3′-C); 153.6 (Ar-4″-C—OCH₃).

### EXAMPLE 4

### Preparation of 1-benzo[d][1,3]dioxol-5-yl-3-[4(4-W-phenyl-substituted)hexahydro-1-pyrazinyl]propane Derivatives

General Procedure

In a 15 mL balloon containing a mixture of 0.129 g of 2-(3,4-methylenedioxophenyl)-1-yl-ethaneyl mesilate (0.50 mMol), 0.074 g of lithium carbonate (1.00 mMol) in acetonitrile (6 mL), was added 1.50 mMol of phenylpiperazine. The mixture was kept in reflux for 24 hours under vigorous agitation and nitrogen atmosphere. At the end of this time the solution was concentrated in rotatory evaporator, solubilized in dichloromethane and mixed in a silica gel. The material was chromatografied in a silica gel column eluted with dichloromethane, followed by chloroform supplying the desired compound.

### 1-benzo[d][1,3]dioxol-5-yl-3-(4-phenylhexahydro-1-pyrazinyl)propane

yellow liquid, 0.158 g, (98%), Rf=0.46 (CHCl₃:EtOH 5%)

RMN ¹H (200 MHz, CDCl₃): δ 1.80 (m, 2H, ArCH₂CH₂CH₂N) 2.40 (m, 2H, ArCH₂CH₂CH₂N); 2.56 (m, 2H, ArCH₂CH₂CH₂N); 2.59 (m, 4H, ArNCH₂CH₂N); 3.20 (m, 4H, ArNCH₂CH₂N); 5.90 (s, 2H, OCH₂O); 6.63 (dd, J²=7.82 Hz, J³=1.70, 1H, Ar—H-6′); 6.69 (d, J³=1.70 Hz 1H, Ar—H-2′); 6.72 (d, J²=7.82 Hz, 1H, Ar—H-5′); 6.87 (m, 3H, N—Ar—H-2″, 4″e 6″); 7.25 (m, 2H, N—Ar—H-3″ e 5″).

RMN ¹³C (50 MHz, CDCl₃): δ 28.5 (NCH₂CH₂CH₂Ar); 33.1 (NCH₂CH₂CH₂Ar); 48.8 (ArNCH₂CH₂N); 53.1 (ArNCH₂CH₂N); 57.5 (NCH₂CH₂CH₂Ar); 100.4 (OCH₂O); 107.8 (Ar-2′-CH); 108.6 (Ar-5′-CH); 115.7 (Ar-2″ e 6″-2CH); 119.5 (Ar-6′-CH); 120.7 (Ar-4″-CH); 128.8 (Ar-3″ e 5″-2CH); 135.6 (Ar-1′-C); 145.3 (Ar-4′-C); 147.3 (Ar-3′-C); 151.0 (Ar-1″-C—N).

### 1-benzo[d][1,3]dioxol-5-yl-3-[4-(4-fluorophenyl)hexahydro-1-pyrazinyl]propane

yellow liquid, 0.168 g (98%.), Rf=0.45 (CHCl₃:EtOH 5%)

RMN ¹H (200 MHz, CDCl₃): δ 1.81 (m, 2H, ArCH₂CH₂CH₂N) 2.41 (m, 2H, ArCH₂CH₂CH₂N); 2.57 (m, 2H, ArCH₂CH₂CH₂N); 2.59 (m, 4H, ArNCH₂CH₂N); 3.13 (m, 4H, ArNCH₂CH₂N); 5.90 (s, 2H, OCH₂O); 6.64 (dd, J²=7.88 Hz, J³=1.65, 1H, Ar—H-6′); 6.70 (d, J³=1.65 Hz 1H, Ar—H-2′); 6.73 (d, J²=7.88 Hz, 1H, Ar—H-5′); 6.87 (m, 2H, N—Ar—H-2″, e 6″); 6.96 (m, 2H, N—Ar—H-3″ e 5″).

RMN ¹³C (50 MHz, CDCl₃): δ 28.5 (NCH₂CH₂CH₂Ar); 33.1 (NCH₂CH₂CH₂Ar); 49.9 (ArNCH₂CH₂N); 52.9 (ArNCH₂CH₂N); 57.5 (NCH₂CH₂CH₂Ar); 100.5 (OCH₂O); 107.8 (Ar-2′-CH); 108.6 (Ar-5′-CH); 115.4 (Ar-3″ e 5″-2CH); 117.4 (Ar-2″ e 6″-2CH); 120.8 (Ar-6′-CH); 135.6 (Ar-1′-C); 145.3 (Ar-4′-C); 147.3 (Ar-3′-C); 147.7 (Ar-1″-C—N); 154.5 (Ar-4″-C—F); 159.2 (Ar-4″-C—F).

### 1-benzo[d][1,3]dioxol-5-yl-3-[4-(4-chlorophenyl)hexahydro-1-pyrazinyl]propane

Solid white, 0.167 g (93%.), Rf=0.50 (CHCl₃:EtOH 5%), p.f.: 72-74° C.

RMN ¹H (200 MHz, CDCl₃): δ 1.79 (m, 2H, ArCH₂CH₂CH₂N) 2.39 (m, 2H, ArCH₂CH₂CH₂N); 2.57 (m, 2H, ArCH₂CH₂CH₂N); 2.59 (m, 4H, ArNCH₂CH₂N); 3.15 (m, 4H, ArNCH₂CH₂N); 5.91 (s, 2H, OCH₂O); 6.63 (dd, J²=7.88 Hz, J³=1.65, 1H, Ar—H-6′); 6.69 (d, J³=1.65 Hz 1H, Ar—H-2′); 6.73 (d, J²=7.88 Hz, 1H, Ar—H-5′); 6.82 (m, 2H, N—Ar—H-2″, e 6″); 7.18 (m, 2H, N—Ar—H-3″ e 5″).

RMN ¹³C (50 MHz, CDCl₃): δ 28.6 (NCH₂CH₂CH₂Ar); 33.2 (NCH₂CH₂CH₂Ar); 49.0 (ArNCH₂CH₂N); 52.9 (ArNCH₂CH₂N); 57.5 (NCH₂CH₂CH₂Ar); 100.4 (OCH₂O); 107.9 (Ar-2′-CH); 108.7 (Ar-5′-CH); 117.0 (Ar-2″ e 6″-2CH); 120.9 (Ar-6′-CH); 124.2 (Ar-4″-C-Cl); 128.7 (Ar-3″ e 5″-2CH); 135.7 (Ar-1′-C); 145.4 (Ar-4′-C); 147.3 (Ar-3′-C); 149.8 (Ar-1″-C—N).

### 1-benzo[d][1,3]dioxol-5-yl-3-[4-(2-methoxyphenyl)hexahydro-1-pyrazinyl]propane

Light yellow liquid, 0.131 g (97%), Rf=0.55 (CHCl₃:EtOH 5%),

RMN ¹H (200 MHz, CDCl₃): δ 1.83 (m, 2H, ArCH₂CH₂CH₂N) 2.44 (m, 2H, ArCH₂CH₂CH₂N); 2.59 (m, 2H, ArCH₂CH₂CH₂N) 2.67 (m, 4H, ArNCH₂CH₂N); 3.12 (m, 4H, ArNCH₂CH₂N); 3.75 (s, 3H, OCH₃); 5.91 (s, 2H, OCH₂O); 6.64 (d, J²=7.78 Hz, J³=1.64, 1H, Ar—H-6′); 6.70 (m, 1H, Ar—H-2′); 6.73 (d, J²=7.78 Hz, 1H, Ar—H-5′); 6.86 (d, J²=7.51 Hz 1H, N—Ar—H-6″); 6.97 (m, 2H, N—Ar—H-3″, 4″ e 5″).

RMN ¹³C (50 MHz, CDCl₃): δ 28.5 (NCH₂CH₂CH₂Ar); 33.2 (NCH₂CH₂CH₂Ar); 50.3 (ArNCH₂CH₂N); 53.2 (ArNCH₂CH₂N); 55.1 (Ar-4″-C—OCH₃); 57.6 (NCH₂CH₂CH₂Ar); 100.5 (OCH₂O); 107.8 (Ar-2′-CH); 108.6 (Ar-5′-CH); 111.0 (Ar-3″-CH) 118.0 (Ar-6″-CH); 120.8 (Ar-5″ e Ar-6′-2CH); 122.6 (Ar-4″-CH); 135.7 (Ar-1′-C); 141.1(Ar-1″-C—N); 145.3 (Ar-4′-C) 147.3 (Ar-3′-C); 152.0 (Ar-2″-C—OCH₃).

### 1-benzo[d][1,3]dioxol-5-yl-3-[4-(4-methoxyphenyl)hexahydro-1-pyrazinyl]propane

Solid light yellow, 0.168 g (95%), Rf=0.41(CHCl₃:EtOH 5%), p.f.: 71-73° C.

RMN ¹H (200 MHz, CDCl₃): δ 1.80 (m, 2H, ArCH₂CH₂CH₂N) 2.40 (m, 2H, ArCH₂CH₂CH₂N); 2.58 (m, 6H, ArCH₂CH₂CH₂N (2H) e ArNCH₂CH₂N, (4H)); 3.09 (m, 4H, ArNCH₂CH₂N); 3.75 (s, 3H, OCH₃); 5.91 (s, 2H, OCH₂O); 6.63 (d, J²=7.88 Hz, 1H, Ar—H-6′); 6.68 (m, 1H, Ar—H-2′); 6.72 (d, J²=7.88 Hz, 1H, Ar—H-5′); 6.82 (m, 2H, N—Ar—H-2″, e 6″); 6.90 (m, 2H, N—Ar—H-3″ e 5″).

RMN ¹³C (50 MHz, CDCl₃): δ 28.7 (NCH₂CH₂CH₂Ar); 33.2 (NCH₂CH₂CH₂Ar); 50.4 (ArNCH₂CH₂N); 53.2 (ArNCH₂CH₂N); 55.4 (Ar-4″-C—OCH₃); 57.6 (NCH₂CH₂CH₂Ar); 100.5 (OCH₂O); 107.9 (Ar-2′-CH); 108.7 (Ar-5′-CH); 114.2 (Ar-3″ e 5″-2CH); 117.9 (Ar-2″ e 6″-2CH); 120.9 (Ar-6′-CH); 135.8 (Ar-1′-C); 145.6 (Ar-4′-C e Ar-1″-C); 147.3 (Ar-3′-C); 153.6 (Ar-4″-C—OCH₃).

### 1-benzo[d][1,3]dioxol-5-yl-3-[4-(2-pyrimidinyl)hexahydro-1-pyrazinyl]propane

Yellowish liquid 0.160 g (98%), Rf=0.35 (CHCl₃:EtOH 5%)

RMN ¹H (200 MHz, CDCl₃): δ 1.80 (m, 2H, ArCH₂CH₂CH₂N) 2.40 (m, 2H, ArCH₂CH₂CH₂N); 2.58 (m, 2H, ArCH₂CH₂CH₂N e 4H, ArNCH₂CH₂N); 3.09 (m, 4H, ArNCH₂CH₂N); 5.90 (s, 2H, OCH₂O); 6.46 (m, 1H, N—Ar—H4″) 6.63 (dd, J²=7.88 Hz, J³=2.20, 1H, Ar—H-6′); 6.68 (d, J³=2.20 Hz 1H, Ar—H-2′); 6.72 (d, J²=7.88 Hz, 1H, Ar—H-5′); 8.29 (m, 2H, N—Ar—H-3″ e 5″).

RMN ¹³C (50 MHz, CDCl₃): δ 28.5 (NCH₂CH₂CH₂Ar); 33.1 (NCH₂CH₂CH₂Ar); 57.2 (ArNCH₂CH₂N); 43.5 (ArNCH₂CH₂N); 57.5 (NCH₂CH₂CH₂Ar); 100.5 (OCH₂O); 107.8 (Ar-2′-CH); 108.6 (Ar-5′-CH); 115.7 (Ar-2″ e 6″-2CH); 120.5 (Ar-6′-CH); 120.7 (Ar-4″-CH); 128.8 (Ar-3″ e 5″-2CH); 135.6 (Ar-1′-C); 145.3 (Ar-4′-C); 147.3 (Ar-3′-C); 151.0 (Ar-1″-C—N).

### EXAMPLE 5

### Preparation of 1-benzo[d][1,3]dioxol-5-yl-4-[4(4-W-phenyl-substituted)hexahydro-1-pyrazinyl]butane Derivatives

General Procedure

In a 15 mL balloon containing a mixture of 0.123 g of 3-(3,4-methylenedioxophenyl)-1-yl-propanyl mesilate (0.45 mMol), 0.074 g of lithium carbonate (1.00 mMol) in acetonitrile (6 mL), were added 1.30 mMol of phenylpiperazine. The mixture was kept in reflux for 24 hours under vigorous agitation and nitrogen atmosphere. At the end of this time the solution was concentrated in a rotatory evaporator, solubilized in dichloromethane and mixed in a silica gel. The material was chromatografied in a silica gel column eluted with dichloromethane, followed by chloroform supplying the desired compound.

### 1-benzo[d][1,3]dioxol-5-yl-4-(4-phenylhexahydro-1-pyrazinyl)butane

solid beige, 0.142 g (93%), Rf=0.47 (CHCl₃:EtOH 5%), p.f.: 53-54° C.

RMN ¹H (200 MHz, CDCl₃): δ 1.59 (m, 4H, ArCH₂CH₂CH₂CH₂N) 2.39 (m, 2H, ArCH₂CH₂CH₂CH₂N); 2.56 (m, 2H, ArCH₂CH₂CH₂CH₂N); 2.58 (m, 4H, ArNCH₂CH₂N); 3.20 (m, 4H, ArNCH₂CH₂N); 5.90 (s, 2H, OCH₂O); 6.62 (dd, J²=7.87 Hz, J³=1.65 Hz, 1H, Ar—H-6′); 6.67 (d, J³=1.65 Hz, 1H, Ar—H-2′); 6.72 (d, J²=7.87 Hz, 1H, Ar—H-5′); 6.82 (m, 1H, Ar—H-4″); 6.93 (m, 2H, N—Ar—H-2″, e 6″); 7.27 (m, 2H, N—Ar—H-3″ e 5″).

RMN ¹³C (50 MHz, CDCl₃): δ 26.1 (NCH₂CH₂CH₂CH₂Ar) 29.4 (NCH₂CH₂CH₂CH₂Ar); 35.3 (NCH₂CH₂CH₂CH₂Ar); 48.9 (ArNCH₂CH₂N); 53.1 (ArNCH₂CH₂N); 58.3 (NCH₂CH₂CH₂CH₂Ar); 100.5 (OCH₂O); 107.8 (Ar-2′-CH); 108.6 (Ar-5′-CH); 115.8 (Ar-2″ e 6″-2CH); 119.4 (Ar-6′-CH); 120.7 (Ar-4″-CH); 128.8 (Ar-3″ e 5″-2CH); 136.0 (Ar-1′-C); 145.3 (Ar-4′-C); 147.3 (Ar-3′-C); 151.1 (Ar-1″-C—N).

### 1-benzo[d][1,3]dioxol-5-yl-4-[4-(4-fluorophenyl)hexahydro-1-pyrazinyl]butane

Yellowish solid, 0.157 g, (98%), Rf=0.44 (CHCl₃:EtOH 5%), p.f.: 53-54° C.

RMN ¹H (200 MHz, CDCl₃): δ 1.57 (m, 4H, ArCH₂CH₂CH₂CH₂N) 2.39 (m, 2H, ArCH₂CH₂CH₂N); 2.56 (m, 6H, ArCH₂CH₂CH₂N (2H) e ArNCH₂CH₂N, (4H)); 3.11 (m, 4H, ArNCH₂CH₂N); 5.90 (s, 2H, OCH₂O); 6.61 (dd, J²=7.87 Hz, J³=1.65, 1H, Ar—H-6′); 6.67 (d, J³=1.65 Hz 1H, Ar—H-2′); 6.72 (d, J²=7.87 Hz, 1H, Ar—H-5′); 6.85 (m, 3H, N—Ar—H-2″, e 6″); 6.95 (m, 2H, N—Ar—H-3″ e 5″).

RMN ¹³C (50 MHz, CDCl₃): δ 26.2 (NCH₂CH₂CH₂CH₂Ar) 29.4 (NCH₂CH₂CH₂CH₂Ar); 35.3 (NCH₂CH₂CH₂CH₂Ar); 49.9 (ArNCH₂CH₂N); 53.1 (ArNCH₂CH₂N); 58.3 (NCH₂CH₂CH₂CH₂Ar); 100.5 (OCH₂O); 107.8 (Ar-2′-CH); 108.6 (Ar-5′-CH); 115.2 (Ar-3″ e 5″-2CH); 117.4 (Ar-2″ e 6″-2CH); 120.9 (Ar-6′-CH); 136.1 (Ar-1′-C); 145.3 (Ar-4′-C); 147.3 (Ar-3′-C); 147.8 (Ar-1″-C—N); 154.5 (Ar-4″-C—F); 159.3 (Ar-4″-C—F).

### 1-benzo[d][1,3]dioxol-5-yl-4-[4-(4-chlorophenyl)hexahydro-1-pyrazinyl]butane

Yellowish solid, 0.145 g (87%), Rf=0.52 (CHCl₃:EtOH 5%), p.f.: 95-96° C.

RMN ¹H (200 MHz, CDCl₃): δ 1.57 (m, 4H, ArCH₂CH₂CH₂CH₂N) 2.38 (m, 2H, ArCH₂CH₂CH₂N); 2.53 (m, 2H, ArCH₂CH₂CH₂N); 2.55 (m, 4H, ArNCH₂CH₂N); 3.14 (m, 4H, ArNCH₂CH₂N); 5.90 (s, 2H, OCH₂O); 6.60 (dd, J²=7.78 Hz, J³=1.65, 1H, Ar—H-6′); 6.66 (d, J³=1.65 Hz 1H, Ar—H-2′); 6.71 (d, J²=7.78 Hz, 1H, Ar—H-5′); 6.81 (m, 3H, N—Ar—H-2″, e 6″); 7.18 (m, 2H, N—Ar—H-3″ e 5″).

RMN ¹³C (50 MHz, CDCl₃): δ 26.1 (NCH₂CH₂CH₂CH₂Ar) 29.4 (NCH₂CH₂CH₂CH₂Ar); 35.3 (NCH₂CH₂CH₂CH₂Ar); 48.9 (ArNCH₂CH₂N); 52.9 (ArNCH₂CH₂N); 58.3 (NCH₂CH₂CH₂CH₂Ar); 100.5 (OCH₂O); 107.9 (Ar-2′-CH); 108.6 (Ar-5′-CH); 116.9 (Ar-2″ e 6″-2CH); 120.9 (Ar-6′-CH); 124.2 (Ar-4″-C-Cl); 128.7 (Ar-3″ e 5″-2CH); 136.0 (Ar-1′-C); 145.3 (Ar-4′-C); 147.3 (Ar-3′-C); 149.7 (Ar-1″-C—N).

### 1-benzo[d][1,3]dioxol-5-yl-4-[4-(4-methoxyphenyl)hexahydro-1-pyrazinyl]butane

solid light beige, 0.139 (84%), Rf=0.40 (CHCl₃:EtOH 5%), p.f.: 76-77° C.

RMN ¹H (200 MHz, CDCl₃): δ 1.56 (m, 4H, ArCH₂CH₂CH₂CH₂N) 2.39 (m, 2H, ArCH₂CH₂CH₂N); 2.55 (m, 2H, ArCH₂CH₂CH₂N); 2.58 (m, 4H, ArNCH₂CH₂N); 3.08 (m, 4H, ArNCH₂CH₂N); 3.75 (s, 3H, OCH₃); 5.90 (s, 2H, OCH₂O); 6.60 (dd, J²=7.78 Hz, J³=1.55 Hz, 1H, Ar—H-6′); 6.66 (d, J³=1.55 Hz 1H, Ar—H-2′); 6.71 (d, J²=7.78 Hz, 1H, Ar—H-5′); 6.82 (m, 3H, N—Ar—H-2″, e 6″); 6.89 (m, 2H, N—Ar—H-3″ e 5″).

RMN ¹³C (50 MHz, CDCl₃): δ 26.2 (NCH₂CH₂CH₂CH₂Ar) 29.4 (NCH₂CH₂CH₂CH₂Ar); 35.3 (NCH₂CH₂CH₂CH₂Ar); 50.4 (ArNCH₂CH₂N); 53.2 (ArNCH₂CH₂N); 55.4 (Ar-4″-C—OCH₃); 58.3 (NCH₂CH₂CH₂CH₂Ar); 100.5 (OCH₂O); 107.8 (Ar-2′-CH); 108.6 (Ar-5′-CH); 114.2 (Ar-3″ e 5″-2CH); 117.9 (Ar-2″ e 6″-2CH); 120.8 (Ar-6′-CH); 136.1 (Ar-1′-C); 145.3 (Ar-4′-C) 145.5 (Ar-1″-C); 147.3 (Ar-3′-C); 153.5 (Ar-4″-C—OCH₃).

### 1-benzo[d][1,3]dioxol-5-yl-4-[4-(2-pyrimidinyl)hexahydro-1-pyrazinyl]butane

Yellowish liquid, 0.113 g (74%), Rf=0.36 (CHCl₃:EtOH 5%)

RMN ¹H (200 MHz, CDCl₃): δ 1.57 (m, 4H, ArCH₂CH₂CH₂CH₂N) 2.39 (m, 2H, ArCH₂CH₂CH₂CH₂N); 2.56 (m, 2H, ArCH₂CH₂CH₂CH₂N); 2.50 (m, 4H, ArNCH₂CH₂N); 3.52 (m, 4H, ArNCH₂CH₂N); 5.90 (s, 2H, OCH₂O); 6.60 (dd, J²=7.78 Hz, J³=1.55 Hz, 1H, Ar—H-6′); 6.67 (d, J³=1.55 Hz 1H, Ar—H-2′); 6.70 (d, J²=7.78 Hz, 1H, Ar—H-5′); 6.46 (m, 1H, Ar—H-4″); 8.27 (m, 2H, N—Ar—H-3″ e 5″).

RMN ¹³C (50 MHz, CDCl₃): δ 26.1 (NCH₂CH₂CH₂CH₂Ar) 29.4 (NCH₂CH₂CH₂CH₂Ar); 35.3 (NCH₂CH₂CH₂CH₂Ar); 57.9 (ArNCH₂CH₂N); 45.3 (ArNCH₂CH₂N); 56.3 (NCH₂CH₂CH₂CH₂Ar); 100.5 (OCH₂O); 107.9 (Ar-2′-CH); 108.6 (Ar-5′-CH); 109.4 (Ar-4″-C—H); 120.9 (Ar-6′-CH); 136.0 (Ar-1′-C); 145.3 (Ar-4′-C); 147.3 (Ar-3′-C); 157.7 (Ar-3″ e 5″-2CH); 161.7 (Ar-1″-C);

The compounds listed above, randomly chosen, were analyzed by H¹-RMN and ¹³C-RMN.

The compounds of formula (I) were spectroscopically characterized and pharmacologically evaluated in functional studies in isolated organs (in vitro) and in pre-clinical assays, by several assays, that are described in details below.

Methodologies Used for Bioassays

In Vitro Isometric Contraction Assay

Vasoconstriction Assay in Mouse Aorta Induced By Norepinephrine

Aorta segments with endothelium (3 mm), withdrawn from young male rats (250-300 g), were placed in containers containing physiologic solution (NaCl 122 mM, KCl 5 mM, NaHCO₃ 15 mM, glucose 11.5 mM, MgCl₂ 1.2 mM, CaCl₂ 1.25 mM and KH₂PO₄ 1.2 mM), kept at 37° C. under aeration with a mixture of 95% of O₂ and 5% of CO₂, and submitted to a pre-charge of 5 mN for 60 minutes. During this period, eventual tension adjustments were performed until the stabilization in 0.5 g, always with renewal with physiologic solution. Then, vasoconstriction was induced by 60 mM KCl solution, in each ring, to verify the viability of the organ on the day of experiment. The preparation was submitted to the contraction with norepinephrine (5×10⁻⁵ M) and, then the rings were washed to reach the basal line (0% of vasoconstriction). The procedure was repeated until the obtainment of a constant curve. Later, a curve, using increasing concentrations of norepinephrine (3×10⁻⁸-5×10⁻⁵ M), was performed for each ring, as a positive control. The segments were washed to reach the basal line (0% of vasoconstriction) and incubated with the test compounds, individually, for 30 minutes. A second norepinephrine curve was performed and obtained data compared to the first control curve. The results were analyzed by GraphSAP Prism software (GraphSAP Software, Inc., San Diego, Calif.). The aorta rings were used once for each compound.

Vasoconstriction Assay in Rabbit Aorta Induced by Phenylephrine

Aorta rings withdrawn from young male rabbits (1500-1700 g) were tensionated to 2 g (20 mN) and kept in Krebs solution, containing 10 mM Hepes, for 30 minutes, under continuous aeration. During this period, eventual tension adjustments were performed until stabilization in 2 g, always with renewal of Krebs solution. Then, vasoconstrictions were induced by 60 mM KCl solution, in each ring, to verify the viability of the organ on the day of the experiment. The preparation was submitted to contraction with phenylephrine (5×10⁻⁵ M) and, then the rings were washed to reach the basal line (0% of vasoconstriction). The procedure was repeated until obtainment of a constant curve. Then, a curve, using increasing concentrations of phenylephrine (5×10⁻⁹-5×10⁻⁴ M), was performed for each ring, as a positive control. The segments were washed to reach the basal line (0% of vasoconstriction) and incubated with the test compounds, individually, for 30 minutes. A second phenylephrine curve was performed and the obtained data compared to the first control curve. The results were analyzed by one-way ANOVA software from Microcal Origin 4.1. The aorta rings were used once for each compound.

In view of the important anti-adrenergic profile evidenced for the new 1-benzo[d][1,3]dioxol-5-yl-2-[4-(2-methoxyphenyl)hexahydro-1-pyrazinyl]ethane derivative (LASSBio 772) in phenylephrine induced vasoconstriction assays in thoracic aorta of rabbit, it was pharmacologically evaluated regarding the affinity in “binding” studies, through the assay in salivary gland, selective for alpha 1A subtypes, and rat liver, selective for alpha 1B subtypes, using [³H]prazosin as ligand.

In Vitro Binding Assay—Homogeneized Tissues

Binding Assay (In Vitro) in Rat Salivary Gland and Liver

The potency of the selected compound, 1-benzo[d][1,3]dioxol-5-yl-2-[4-(2-methoxyphenyl)hexahydro-1-pyrazinyl]ethane (LASSBio 772), was determined through method of an assay based on the use of 96 well microplates MultiScreen-PH 96 (Millipore), with final volume of 200 microL, in which a phosphocellulose filter paper was placed in each lower well following the binding of positive charged substrate after the washing/filtering step.

The test-compound concentrations, LASSBio 772, for obtaining competition curves varied from 3 μM to 100 nM. Thus, 0.1 microg of membrane proteins expressing the rat alpha 1A subtype (salivary gland) or alpha 1B (liver) were added in 50 mM Tris-HCl (pH 7.5) buffer solution to each microplate well. After 60 minutes of incubation at 22° C., the reaction was concluded by fast filtration through glass fiber filters (Packard Instruments CO., Meriden, Conn.). The filters were rinsed 3 times with 3 mL of buffer solution cooled in an ice bath. The radioactivity levels were determined using a liquid scintillation counter in the Top-Count instrument. The competition curves were analyzed with the use of the capability of overlapping curves from GraphSAP Prism software (GraphSAP Software, Inc., San Diego, Calif.).

The rat tissues (salivary gland, liver) after homogenization, centrifugation, re-suspension and re-centrifugation, were incubated with [³H]prazosin (0.06 nM), in 50 mM Tris-HCl (pH 7.5) buffer solution in each microplate well. After 60 minutes of incubation at 22° C., the reaction was concluded by fast filtration (cell Harvesther Brandel, Gaithersburg, Md.) through glass fiber filters and the filters were rinsed 3 times with 3 mL of buffer solution cooled in an ice bath. The radioactivity levels were determined using a liquid scintillation counter in the Top-Count instrument. The competition curves were analyzed with the use of capability of overlapping curves from GraphSAP Prism software (GraphSAP Software, Inc., San Diego, Calif.). The results are expressed as a percentage of the specific affinity control in the presence of LASSBio 772.

For the alpha 1A subtype 0.06 nM [³H]prazosin was used as ligand (reference—WB 4101—IC₅₀ 0.60 nM); for the alpha 1B subtype 0.06 nM [³H]prazosin was used as ligand (reference—spiperone —IC₅₀ 2.0 nM) and 0.10 nM [³H]prazosin as ligand (reference—prazosin—0.10 nM) (Michel et al, Br. J. Pharmacol., 98, 883-889, 1989).

The presented pharmacological results reveal that LASSBio 772 has high affinity for the alpha 1A subtype, with Kᵢ, 0.14 nM, similar to therapeutic agent tamsulosin (Flomax®, Boehringer Ingelheim), whose Kᵢ value corresponds to 0.13 nM (Kuo et al., Bioorg. Med. Chem. Lett., 8, 2263-2275, 2000) and thus it has been used to relieve the obstructive symptoms of the benign prostatic hyperplasia (BPH) with uroselective features. Thus, we identified in LASSBio 772, the ability to antagonize the alpha-1 A receptors, showing usefulness for treatment of BPH and for the tissue relaxation of the lower urinary tract in mammals, preferentially humans.

The obtained results revealed significant LASSBio-772 inhibitory potency for the alpha 1A subtype, with IC₅₀ 0.26 nM (reference—WB 4101—IC₅₀ 0.60 nM) and important lower antagonist activity for the alpha 1B subtype, with IC₅₀ 450.00 nM (reference—prazosin 0.10 nM). These data demonstrate the important selectivity for the alpha 1A subtype, whose selectivity index (I.S. alpha 1B/alpha 1A) in comparison to the alpha 1B subtype is 1730 times, corroborating the antagonist properties for BPH's therapeutic use.

Additionally, considering Tamsulosin, this makes evident the selectivity index (I.S._{alpha 1B/alpha 1A}) ca. 14, value of 123.5 times smaller than LASSBio 772, reinforcing its selective properties to the alpha 1A adrenoceptor subtype, involved in the physiopathology of the lower urinary tract symptoms.

Still in this context, tamsulosin (FlomaxÒ, Boehringer Ingelheim) and prazosin (MinipressÒ, Pfizer) exhibit similar homodynamic profiles in rabbit and rat, both having accentuated hypo-tensor effects, which seems to be related to the alpha 1B adrenoceptor antagonism. However, clinically, tamsolusin seems not to produce the uncomfortable adverse effects associated with the quinazolyl antagonist prazosin (Hieble et al., Eur. J. Pharmacol., 373, 51-62, 1999), what some authors suggest is related to its formulation (Blue et al., Br. J. Pharmacol., 120, 107P. 1997, Bock & Patane, Ann. Rep. Med. Chem., 35, 221-230, 2000).

Determination of the LASSBio 772 Antagonist Potency (pKB) on the Vasoconstriction of Rabbit or Rat Thoracic Aorta.

Considering the significant antagonist activity of 30 microM of LASSBio-772 in comparison to the vasoconstriction induced by 5×10⁻⁴ M of phenylephrine, it was decided to determine the apparent antagonist potency (pK_{B}) for this derivative in rabbit or rat thoracic aorta.

In this sense, experiments in rabbits indicated, in preliminary results, that the LASSBio-772 derivative presents apparent antagonist potency around 7.85 (14.1 nM), this result obtained by the potency average of 18 experiments, in concentrations that varied from 3 nM to 300 nM, with a minimum of 3 experiments per concentration, using Schild equation, described below.
pK_{B}=Log(cr−1)−LogB,

where:

cr is the ratio between phenylephrine concentrations in the presence and absence of antagonist; and

B is the antagonist's concentration.

Particularly for 1-benzo[d][1,3]dioxol-5-yl-2-[4-(2-methoxyphenyl) hexahydro-1-pyrazinyl]ethane derivative (LASSBio 772), the obtained results presented significant anti-adrenergic activity, with apparent pK_{B} 7.85 (KB 6.3±0.10 nM—rabbit aorta—subtypes alpha 1A and alpha 1B) and pK_{B} 10.88 (0.013 nM±0.08—rat aorta). The antagonistic potency result of the LASSBio derivative 772 corroborates with the elevated anti-adrenergic profile that is evidenced in binding studies.

Comparison of LASSBio 772 (pK_{B}=10.86 (0.014 nM) with the selective alpha 1D antagonist BMY 7378 (pK_{B}=8.22 (6.025 nM)), described in the patent U.S. Pat. No. 6,326,372 incorporated herein as reference, reveals that LASSBio affinity 772 is, at least, 430 times greater for the same adrenergic receptor subtype, revealing elevated antagonist profile of LASSBio772 to this receptor subtype.

With regard to the antagonist potential considering (alpha) 1D-adrenoceptor, expressed functionally in rat aorta, LASSBio-772 presented high affinity for this receptor subtype, with pK_{B}=10.86 (0.014 nM), presenting an antagonist potency similar to tamsulosin, whose pK_{B}=10.76 (0.018 nM) (Kuo et al., Bioorg. Med. Chem. Lett., 8, 2263-2275, 2000).

These pharmacological results obtained by binding and functional bioassays, prove the rational planning of the compound of formula (I), particularly the LASSBio 772 derivative, which presents selective antagonist profile for alpha 1A/alpha 1D which does not act on the alpha 1B subtypes—subtypes that are the main AR subtypes involved in the pressure regulation in humans starting at 60 years old, whose antagonism is related to undesirable hypotension effects (Rudner, et al., Circulation, 100, 2336-2343, 1999). In this sense, the alpha 1A/alpha 1D selective antagonist profile exhibited by the LASSBio 772 derivative is in concordance with studies in the literature which suggest the development of selective alpha 1A/alpha 1D antagonists that would indeed relieve both obstructive and irritating symptoms of BPH, action mechanism maybe associated to the tamsulosin pharmacological profile (Michelotti et al. Pharmacol. Ther., 88, 281-309, 2000).

Selectivity Assay for Protein G-Coupled, Beta-Adrenergic, Muscarinic Cholinergic and Histaminergic Bioreceptors

Determination of the Inhibitory Potency (IC₅₀) of LASSBio 772 on Vasoconstriction of the Cobaio (Guinea Pig) Trachea

Cobaios albinos from both sexes, weighing between 400 and 600 g were used in this experiment. The animals were sacrificed by cervical displacement and posterior bleed. Then, their tracheas were removed, kept in 5 microM indometacin added Krebs-Henseleit physiologic solution and oxygenated with carbogenic mixture (95% O₂ and 5% CO₂), proceeding the removal of the connective and adhered adipose tissue. From these tracheas were obtained segments with 4-6 rings, that are sectioned in the opposite side of the smooth musculature, originating strips used in the contractions assay (ARAKIDA et al., J. Pharmacol. Exper. Therap., 287: 633-639, 1998).

These strips were adapted in glass containers of 1.0 mL containing nutritious solution and continuous aeration with carbogenic mixture. The containers are maintained in a bath (Ugo Basile 4050) at the constant temperature of 37±0.50° C., mimicking thus physiologic terms in vitro. The trachea strips were connected to isometric force transducers (Ugo Basile 7011-4), submitted to a tension of 1 g and rested for 1 hour, changing the solution in 15 minute intervals, to obtain the balance to this tension.

After the period, the organ is treated with agonists that will induce contractions. These will be transduced, amplified and registered in a potenciometric recorder (Lineal Records 1201). The tissue viability was evaluated by contraction induction with histamine 100 microM or carbacol 10 microM. The agonists used in selectivity study of the compound LASSBio 772 were histamine 100 microM, salbutamol (10⁻¹⁰-3.3×10⁻⁵ M) and carbacol (10 mM).

In accomplishment of the assays with the compound LASSBio 772, the control curve with said agonist was initially obtained. Then, the curves of the agonist with the vehicle (DMSO) and compound LASSBio 772 were obtained. The preparation was incubated with the compound and the vehicle for 30 minutes, and soon after the contraction was induced by the agonist.

The trachea preparations were only used on the day of the extraction, having about 12 hours usability. At the end of each experiment, tracheas were contracted with said agonist, to evaluate if the tissues were still viable.

The answers were expressed in % of contraction, considering 100% of contraction that one obtained with the agonist in the presence of the vehicle. The volumes administrated in the containers did not exceed 0.1% of the total volume.

The compound LASSBio 772 was evaluated in the concentration of 30 microM as for its relaxation capability in comparison to contractions induced by carbacol, muscarinic cholinergic agonist, at a 10 microM concentration. In these experiments the compound was not able to significantly inhibit the contractions (n=3, % of Contraction=122.1±8.6).

In another experiment the compound was evaluated for its capability to inhibit the relaxation produced by salbutamol (10⁻¹⁰-3.3×10⁻⁵ M), beta 2 adrenergic agonist, in strips of cobaio trachea pre-contracted with carbacol (10 microM). In this experiment the compound also was not able to inhibit significantly the relaxing effect of salbutamol (n=4).

However, the evaluation of this compound in comparison to the contractions induced by histamine (100 microM), H₁ receptors agonist, the compound LASSBio 772 (3 nM-30 microM), was able to significantly inhibit contractions, showing an IC₅₀=309 nM (IC₅₀=concentration able to inhibit 50% of the effect).

These results corroborate with a good profile of alpha adrenergic antagonist selectivity, dissociated of effects of other receptors, for the compound LASSBio 772, having in mind that it did not present any activity in comparison to the muscarinic cholinergic and/or beta 2 adrenergic receptors. Besides the observed effect in comparison to H₁ receptors, it is valid to stress that alpha-adrenergic antagonist evaluation yielded an IC₅₀ 0.26 nM, while its H₁ anti-histaminergic effect yielded an IC₅₀ 309 nM, that gives a selectivity ratio of about 1000 times for the alpha-adrenergic antagonist effect in relation to the H1 antagonist effect.

Binding Assay (In Vitro) in Human Prostatic Tissues

Binding Assays of [³H]-prazosin:

The human prostatic membranes preparations are obtained according to the protocol described by Wong and collaborators (J. Med. Chem., 41, 2643-2650, 1998) and Muramatsu and collaborators (Br. J. Urol., 74, 572-578, 1994). For the experiment (150 μg protein/tube), [³H]-prazosin 0.3 nM was used in the presence and absence of pharmaceuticals (0.1-100 μM), incubated for 45 min in medium containing 50 mM Tris-HCl, 1 mM EDTA (pH 7.4) at 25° C. The reaction is ended by the addition of cold buffer (4×5 ml), followed by vacuum filtration in glass fiber filters (GF/C), and the nonspecific binding defined by addition of cold prazosin (1 μM). The radioactivity retained in the filter is determined through liquid scintillation spectrometry. The protein dosage follows the method described by Lowry et al (1951). In the case of existing antagonism in these receptors the addition of increasing concentrations of pharmaceuticals dislocates the specific binding of [³H]-prazosin to the alpha-adrenergic receptor, from which it is calculated the inhibitory potency through IC₅₀ (pharmaceutical concentration that inhibits the specific binding in 50%).

The preliminary results demonstrated high affinity of LASSBio 772 for the prostatic tissue with antagonist potency, pK_{B} 9.30 (0.5 nM), corroborating its potential use as an antagonist for the prostatic obstructive symptoms modulated by the alpha 1A subtype. The comparisons of affinities in similar models with tamsulosin (pK_{B}, 9.68 (0.2 nM), KMD-3213 (pK_{B}, 9.45 (0.35 nM) and prazosin (pK_{B}, 8.84 (1.45 nM) (Motiyama et al. Eur. J. Phar., 331: 39-42, 1997) corroborate the use of LASSBio 772 in the treatment of lower urinary tract symptoms like the Benign Prostatic Hyperplasia.

Statistical Analysis

All the results were analyzed statistically by the Student “t” test for a level of significance *p<0.05. The results were expressed on an average±average standard deviation using Sigma Stat version 1.0 software.

IC₅₀ values were obtained through the non-linear sigmoid regression using Microcal Origin 4.1 software.

Determination of the IC₅₀: IC₅₀ values (concentration responsible for half the maximum inhibition effect controlled by specific ligand) and Hill coefficients (nh) were determined through non-linear regression analysis of the competition curves using the overlapping curve of the Hill equation. Experimental data were analyzed by GraphSAP Prism software.

Kᵢ calculation: The inhibition constants (Kᵢ) were calculated by the Cheng Prussof equation, shown below:
(Kᵢ=IC₅₀/(1+L/K_{D})

Where:

L=radioligand concentration used in the assay

K_{D}=radioligand affinity for the receptor

Following the method described above, a representative compound of the formula (I) of the invention, LASSBio 772, showed significant anti-adrenergic activity for the alpha 1A and alpha 1D subtypes, being the selectivity index in comparison to alpha 1B subtype of, at least, 1700 for the alpha 1A subtype and 10000 for the alpha 1D subtype.

The obtained results were grouped in the table I as follows.

TABLE 1
Pharmacological data of LASSBio-772 potency and affinity
based on functional and “binding” affinity studies using native alpha-
adrenoceptors

| | | | | |
|---|---|---|---|---|
| Functional Studies | pK_{B} | K_{B} (nM) | [95% C.I.] | n |

Rabbit aorta - α_{1A} > α_{1B}

| | | | | |
|---|---|---|---|---|
| Prazosin^{a} | 9.05 | 1.02 | [0.95-1.10] | >20 |
| LASSBio 772 | 7.85^{b} | 14.12 | — | 18 |

Rat aorta - α_{1D}

| | | | | |
|---|---|---|---|---|
| Prazosin | 9.77 | 0.17 | [9.65-9.88] | 3 |
| BMY7378 | 8.22^{c} | 6.025 | — | — |
| LASSBio 772 | 10.86^{d} | 0.014 | [10.33-11.39] | 10 |
| LASSBio 772B | 10.70^{d} | 0.020 | [10.52-10.88] | 3 |

Human Prostate - α_{1A}

| LASSBio 772 | 9.3/0.5 | — | 2 | |
|---|---|---|---|---|
| “Binding” studies | IC₅₀ | Kᵢ (nM) | [95% C.I.] | N |

Rat salivary gland - α_{1A}

| | | | | |
|---|---|---|---|---|
| LASSBio 772 | 0.26 | 0.14 | — | 2 |

Rabbit liver - α_{1A}

| | | | |
|---|---|---|---|
| LASSBio 772 | 4.9 | 4.00-5.00 | 3 |
| LASSBio 772B | 23.9 | 19.00-29.00 | 3 |

Rat liver - α_{1B}

| | | | | |
|---|---|---|---|---|
| LASSBio 772 | 450.00 | — | [140-1380] | 3 |
| LASSBio 772B | 130.00 | — | [100-180] | 3 |

Literature (Yamagishi et al., Eur. J. Pharmacol., 315, 73-79, 1996); b) apparent pK_{B}, n = 18; c)
Literature (Carroll et al, Bioorg. Med. Chem. Lett., 11, 1119-1121, 2001); d) P < 0.05 regarding prazosin

It is important to emphasize the structural simplicity of LASSBio 772, which does not have asymmetric center and, therefore, is considered a pure substance, free from safety problems pertinent to the clinical administration and complications in the synthesis and, mainly, of purification associated with chiral substances like tamsulosin and other previously available molecules. Additionally, LASSBio 772 is showed to be selective for alpha 1A/alpha 1D adrenoceptors, whose selectivity indexes for the alpha 1B subtype and the beta 2, H1 and cholinergic muscarinic receptors, of at least a thousand times, gives to this agent a therapeutic window recommended for its clinical use, exempting it of putative side effects associated to these other protein-G coupled transmembrane receptors.

Animal and Homodynamic Measurements:

The experiments were performed with male rabbits weighing 3 kg, from the CECAL (Centro de Criação de Animais de Laboratório) at Fundação Oswaldo Cruz. The animals were anesthetized with sodium pentobarbital (40 mg/kg) in the ear marginal vein, and anesthesia maintenance was obtained with supplemental intravenous (i.v.) doses of 5 mg/kg as necessary. The rats were tracheomized, inserted with a polyethylene canule, and immobilized with pancuronium bromide (1 mg/kg, i.v.), with supplemental doses of 0.2 mg/kg for each hour of experiment and artificially ventilated with a mechanical fan (Rodent ventilator 7025, Ugo Basile) with a volume of 10 ml/kg and frequency from 40 to 50 incursions/min. The right femoral vein was catheterized for drug administration.

The arterial pressure was monitored through abdominal aorta catheter via the right femoral artery and connected to a quartz transductor Hewlett Packard (1290 A), which was connected to an arterial pressure processor and recorder Hewlett Packard (system 7754 with amplifier 8805 B).

The systolic (SAP) and diastolic (DAP) arterial pressures were obtained directly from the records and the average arterial pressure (AAP) was calculated by DAP's Sum with a third of the differential pressure [AAP=DAP+(SAP−DAP/3)]. The cardiac frequency was determined by counting the pressure waves, visualized with the speed increase of the recorder paper.

The cardiac debit (DC) was monitored using an electromagnetic probe, connected to a Skalar flowmeter model MDL 1401. After left thoraxatomy, between the second and third ribs, was made an incision in the pericardium and the heart was exposed to enable the visualization and dissection of the ascending aorta for installation of the electromagnetic probe. The systemic vascular resistance (SVR) was calculated as a quotient of the average arterial pressure by the cardiac debit, multiplied by a conversion factor 80 (SVR=AAP/DC×80) and expressed in dyn/seg/cm-5.

At the conclusion of the anesthetic-surgical procedures, a 15-minute period was allowed for stabilization of the homodynamic parameters. Before any pharmacological manipulation the average arterial pressure, cardiac frequency and cardiac debit measurements were obtained. These values were considered basal homodynamic values.

Dose-Response Curve of the Phenylephrine Vasopressor Effect:

After surgical procedures and stabilization of the homodynamic parameters, a dose-response curve of the phenylephrine pressure effects was performed (0.1-100 mg/kg, i.v.), that were named control curves.

Studies of the Inhibitory Effects of LASSBio 772B on Phenylephrine Vasopressor Effects:

After the control curve of phenylephrine, the animals were treated with LASSBio 772B in doses of (1-100 μg/kg, i.v.). Then, a new dose-response curve of phenylephrine was performed (0.1-100 mg/kg, i.v.).

Statistical Analysis

The results were expressed as average±standard deviation (E.P.M.) The administration effects of LASSBio 772 with regard to the basal values were analyzed through variance analysis (ANOVA) for repeated measurements. When a significant statistical difference was found in the test ANOVA, the test Newman-Keuls was used to locate the differences. The paired t test was used to analyze the phenylephrine pressure effects before and after the treatment with LASSBio 772B. Differences with p<0.05 were considered significant. All the calculations were done on the computer, using the statistic software Instat-2.0.

Arterial Pressure Effects of LASSBio 772 Administration:

The administration of increasing doses of LASSBio 772 (1-100 μg/kg, i.v.) induced a significant hypo-tensor response from the dose of 3 μg/kg, when compared with the basal values. In the largest dose administrated (100 μg/kg, i.v.), AAP's values which were before 90±4 mmHg; of DAP 80±4 mmHg and SAP 110±4 mmHg, they were reduced, in about just 10%, for 79±4 mmHg, 71±4 mmHg and 94±7 mmHg (n=5, p<0.05) respectively.

Inhibitory Effects of LASSBio 772 on Phenylephrine Vasopressor Effects:

The administration of increasing phenylephrine doses (0.1-100 μg/kg, i.v.) induced AAP and SVR increase, to maximum values in the dose of 100 μg/kg. After LASSBio 772 administration, the effects produced by the phenylephrine were significantly blocked when compared with the control curve. AAP and SVR increases that were 34±3% and 136±8%, respectively, in the presence of LASSBio 772B became 9±4% and 15±5%, respectively (n=5, p<0.05) (FIG. 2). The decrease of DC from 44±3% induced by the phenylephrine, was reduced to 7±2% (n=5 p<0.05).

Such results indicate that the compound LASSBio 772 alone slightly reduced the average arterial pressure (10%) and prevented the phenylephrine hypo-tensor effect, without however causing significant clinically hypotension.

1. The use, as alpha adrenergic antagonist selective for the alpha 1A and/or alpha 1D subtypes, of a compound wherein it has the structure according to formula (I): 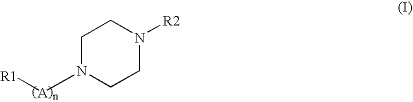
where:
R1 corresponds to 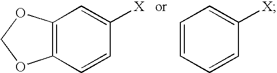
X corresponds to methylene, oxygen, sulfur or nitrogen;
A corresponds to CH₂ or CH(CH₃);
N represents an integer between 1 and 4;
R2 is hydrogen, alkyl, cycloalkyl; furyl, thiophenyl, pyridinyl, pyrimidinyl, pyrrolyl, thiazolyl, quinazolyl, isoquinolyl or phenyl-W;
where W is hydrogen, ortho-alkyl, ortho-cycloalkyl, ortho-alkoxyl, ortho-cycloalkoxyl, ortho-thioxyl, ortho-aryloxyl, ortho-sulfones, ortho-sulphydes, ortho-sulfoxides, ortho-sulfonates, ortho-sulfonamides, ortho-amine, ortho-amide, ortho-halide, ortho-carboalkoxyl, ortho-carbothioalkoxyl, ortho-trihaloalkene, ortho-cyane, ortho-nitro, meta-alkyl, meta-cycloalkyl, meta-alkoxyl, meta-clycloalkoxyl, meta-thioxyl, meta-aryloxyl, meta-sulfones, meta-sulphydes, meta-sulfoxides, meta-sulfonates, meta-sulfonamides, meta-amine, meta-amide, meta-halide, meta-carboalkoxyl, meta-carbothioalkoxyl, meta-trihaloalkane, meta-cyane, meta-nitro, para-alkyl, para-cycloalkyl, para-alkoxyl, para-cycloalkoxyl, para-thioxyl, para-aryloxyl, para-sulfones, para-sulphides, para-sulfoxides, para-sulfonates, para-sulfonamides, para-amine, para-amide, para-halides, para-carboalkoxyl, para-carbothioalkoxyl, para-trihaloalkene, para-cyane, para-nitro,
or its salts, solvates or pharmaceutically acceptable derivatives.

2. The use according to claim 1, wherein said compound is chosen from the group consisting of 1-(2-1,3-Benzodioxol-5-yl-ethyl)-4-(2-methoxy-phenyl)-piperazine and 4-Phenethyl-1-(2-methoxy-phenyl)-piperazine.

3. The use according to claim 1, wherein it is for the production of a medication to treat and/or prevent the lower urinary tract symptoms (LUTS) in mammals.

4. Pharmaceutical composition for the treatment and/or the prevention of the lower urinary tract symptoms (IURS) in mammals comprising a vehicle, diluent and/or pharmaceutically acceptable excipient and at least one compound of formula (I): 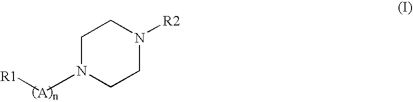
where:
R1 corresponds to 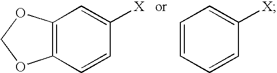
X corresponds to methylene, oxygen, sulfur or nitrogen;
A corresponds to CH₂ or CH(CH₃);
N represents an integer between 1 and 4;
R2 is hydrogen, alkyl, cycloalkyl; furyl, thiophenyl, pyridinyl, pyrimidinyl, pyrrolyl, thiazolyl, quinazolyl, isoquinolyl or phenyl-W;
where W is hydrogen, ortho-alkyl, ortho-cycloalkyl, ortho-alkoxyl, ortho-cycloalkoxyl, ortho-thioxyl, ortho-aryloxyl, ortho-sulfones, ortho-sulfetos, ortho-sulfoxides, ortho-sulfonates, ortho-sulfonamides, ortho-amine, ortho-amide, ortho-halides, ortho-carboalkoxyl, ortho-carbothioalkoxyl, ortho-trihaloalkene, ortho-cyane, ortho-nitro, meta-alkyl, meta-cycloalkyl, meta-alkoxyl, meta-clycloalkoxyl, meta-thioxyl, meta-aryloxyl, meta-sulfones, meta-sulphides, meta-sulfoxides, meta-sulfonates, meta-sulfonamides, meta-amine, meta-amide, meta-halides, meta-carboalkoxyl, meta-carbothioalkoxyl, meta-trihaloalkane, meta-cyane, meta-nitro, para-alkyl, para-cycloalkyl, para-alkoxyl, para-cycloalkoxyl, para-thioxyl, para-aryloxyl, para-sulfones, para-sulphides, para-sulfoxides, para-sulfonates, para-sulfonamides, para-amine, para-amide, para-halides, para-carboalkoxyl, para-carbothioalkoxyl, para-trihaloalkene, para-cyane, para-nitro;
or its salts, solvates or pharmaceutically acceptable derivatives.

5. Pharmaceutical composition according to claim 4, wherein the compound of formula (I) is chosen from the group consisting of 1-(2-1,3-Benzodioxol-5-yl-ethyl)-4-(2-methoxy-phenyl)-piperazine and 4-Phenethyl-1-(2-methoxi-phenyl)-piperazine.

6. Pharmaceutical composition according to claim 4, wherein said lower urinary tract symptom comprises the benign prostatic hyperplasia.

7. (canceled)

8. (canceled)

9. (canceled)

10. The use according to claim 2, wherein it is for the production of a medication to treat and/or prevent the lower urinary tract symptoms (LUTS) in mammals.

11. Use of compounds of formula (I) 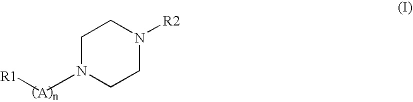
where:
R1 corresponds to 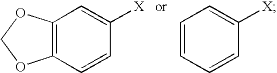
X corresponds to methylene, oxygen, sulfur or nitrogen;
A corresponds to CH₂ or CH(CH₃);
N represents an integer between 1 and 4;
R2 is hydrogen, alkyl, cycloalkyl; furyl, thiophenyl, pyridinyl, pyrimidinyl, pyrrolyl, thiazolyl, quinazolyl, isoquinolyl or phenyl-W;
where:
W is hydrogen, ortho-alkyl, ortho-cycloalkyl, ortho-alkoxyl, ortho-cycloalkoxyl, ortho-thioxyl, ortho-aryloxyl, ortho-sulfones, ortho-sulphides, ortho-sulfoxides, ortho-sulfonates, ortho-sulfonamides, ortho-amine, ortho-amide, ortho-halides, ortho-carboalkoxyl, ortho-carbothioalkoxyl, ortho-trihaloalkene, ortho-cyane, ortho-nitro, meta-alkyl, meta-cycloalkyl, meta-alkoxyl, meta-clycloalkoxyl, meta-thioxyl, meta-aryloxyl, meta-sulfones, meta-sulphides, meta-sulfoxides, meta-sulfonates, meta-sulfonamides, meta-amine, meta-amide, meta-halides, meta-carboalkoxyl, meta-carbothioalkoxyl, meta-trihaloalkane, meta-cyane, meta-nitro, para-alkyl, para-cycloalkyl, para-alkoxyl, para-cycloalkoxyl, para-thioxyl, para-aryloxyl, para-sulfones, para-sulphides, para-sulfoxides, para-sulfonates, para-sulfonamides, para-amine, para-amide, para-halides, para-carboalkoxyl, para-carbothioalkoxyl, para-trihaloalkene, para-cyane, para-nitro;
or its salts, solvates or pharmaceutically acceptable derivatives,
said use characterized for being for the preparation of a medicament for the treatment and/or the prevention of the lower urinary tract symptoms (IURS) in mammals, including humans.
